SEQUENCE LISTING (2 sequences)

SEQ ID NO: 1 - DNA, 13503 bp, Homo sapiens
gtgcgcggcggcggcggcagcggcgtcggctcggggttctccgggagagggggagtgcgcggcggccgcagctgccacaaaccaggtgaagctttgttctaagaatatttgtttcatctagtttatgagtccaaatgatatagactgtaaatgtcacagcagtggtgaaagactgctcggtcatgagcaccgacagtaactcactggcacgtgaatttctgaccgatgtcaaccggctttgcaatgcagtggtccagagggtggaggccagggaggaagaagaggaggagacgcacatggcaacccttggacagtaccttgtccatggtcgaggatttctattacttaccaagctaaattctataattgatcaggcattgacatgtagagaagaactcctgactcttcttctgtctctccttccactggtatggaagatacctgtccaagaagaaaaggcaacagattttaacctaccgctctcagcagatataatcctgaccaaagaaaagaactcaagttcacaaagatccactcaggaaaaattacatttagaaggaagtgccctgtctagtcaggtttctgcaaaagtaaatgtttttcgaaaaagcagacgacagcgtaaaattacccatcgctattctgtaagagatgcaagaaagacacagctctccacctcagattcagaagccaattcagatgaaaaaggcatagcaatgaataagcatagaaggccccatctgctgcatcattttttaacatcgtttcctaaacaagaccaccccaaagctaaacttgaccgcttagcaaccaaagaacagactcctccagatgctatggctttggaaaattccagagagattattccaagacaggggtcaaacactgacattttaagtgagccagctgccttgtctgttatcagtaacatgaacaattctccatttgacttatgtcatgttttgttatctttattagaaaaagtttgtaagtttgacgttaccttgaatcataattctcctttagcagccagtgtagtgcccacactaactgaattcctagcaggctttggggactgctgcagtctgagcgacaacttggagagtcgagtagtttctgcaggttggaccgaagaaccggtggctttgattcaaaggatgctctttcgaacagtgttgcatcttctgtcagtagatgttagtactgcagagatgatgccagaaaatcttaggaaaaatttaactgaattgcttagagcagctttaaaaattagaatatgcctagaaaagcagcctgacccttttgcaccaagacaaaagaaaacactgcaggaggttcaggaagattttgtgttttcaaagtatcgtcatagagcccttcttttacctgagcttttggaaggagttcttcagattctgatctgttgtcttcaaagtgcagcttcaaatcccttctacttcagtcaagccatggatttggttcaagaattcattcagcatcatggatttaatttatttgaaacagcagttcttcaaatggaatggctggttttaagagatggagttcctcccgaggcctcagagcatttgaaagccctaataaatagtgtgatgaaaataatgagcactgtcaaaaaagtgaaatcagagcaacttcatcattcgatgtgtacaagaaaaaggcacagacgatgtgaatattctcattttatgcatcatcaccgagatctctcaggtcttctggtttcggcttttaaaaaccaggtttccaaaaacccatttgaagagactgcagatggagatgtttattatcctgagcggtgctgttgcattgcagtgtgtgcccatcagtgcttgcgcttactacagcaggcttccttgagcagcacttgtgtccagatcctatcgggtgttcataacattggaatatgctgttgtatggatcccaaatctgtaatcattcctttgctccatgcttttaaattgccagcactgaaaaattttcagcagcatatattgaatatccttaacaaacttattttggatcagttaggaggagcagagatatcaccaaaaattaaaaaagcagcttgtaatatttgtactgttgactctgaccaactagcccaattagaagagacactgcagggaaacttatgtgatgctgaactctcctcaagtttatccagtccttcttacagatttcaagggatcctgcccagcagtggatctgaagatttgttgtggaaatgggatgctttaaaggcttatcagaactttgtttttgaagaagacagattacatagtatacagattgcaaatcacatttgcaatttaatccagaaaggcaatatagttgttcagtggaaattatataattacatatttaatcctgtgctccaaagaggagttgaattagcacatcattgtcaacacctaagcgttacttcagctcaaagtcatgtatgtagccatcataaccagtgcttgcctcaggacgtgcttcagatttatgtaaaaactctgcctatcctgcttaaatccagggtaataagagatttgtttttgagttgtaatggagtaagtcaaataatcgaattaaattgcttaaatggtattcgaagtcattctctaaaagcatttgaaactctgataatcagcctaggggagcaacagaaagatgcctcagttccagatattgatgggatagacattgaacagaaggagttgtcctctgtacatgtgggtacttcttttcatcatcagcaagcttattcagattctcctcagagtctcagcaaattttatgctggcctcaaagaagcttatccaaagagacggaagactgttaaccaagatgttcatatcaacacaataaacctattcctctgtgtggcttttttatgcgtaagtaaagaagcagagtctgacagggagtcggccaatgactcagaagatacttctggctatgacagcacagccagcgagcctttaagtcatatgctgccatgtatatctctcgagagccttgtcttgccttctcctgaacatatgcaccaagcagcagacatttggtctatgtgtcgttggatctacatgttgagttcagtgttccagaaacagttttataggcttggtggtttccgagtatgccataagttaatatttatgataatacagaaactgttcagaagtcacaaagaggagcaaggaaaaaaggagggagatacaagtgtaaatgaaaaccaggatttaaacagaatttctcaacctaagagaactatgaaggaagatttattatctttggctataaaaagtgaccccataccatcagaactaggtagtctaaaaaagagtgctgacagtttaggtaaattagagttacagcatatttcttccataaatgtggaagaagtttcagctactgaagccgctcccgaggaagcaaagctatttacaagtcaagaaagtgagacctcacttcaaagtatacgacttttggaagcccttctggccatttgtcttcatggtgccagaactagtcaacagaagatggaattggagttacctaatcagaacttgtctgtggaaagtatattatttgaaatgagggaccatctttcccagtcaaaggtgattgaaacacaactagcaaagcctttatttgatgccctgcttcgagttgccctcgggaattattcagcagattttgaacataatgatgctatgactgagaagagtcatcaatctgcagaagaattgtcatcccagcctggtgatttttcagaagaagctgaggattctcagtgttgtagttttaaacttttagttgaagaagaaggttacgaagcagatagtgaaagcaatcctgaagatggcgaaacccaggatgatggggtagacttaaagtctgaaacagaaggtttcagtgcatcaagcagtccaaatgacttactcgaaaacctcactcaaggggaaataatttatcctgagatttgtatgctggaattaaatttgctttctgctagtaaagccaaacttgatgtgcttgcccatgtatttgagagttttttgaaaattattaggcagaaagaaaagaatgtttttctgctcatgcaacagggaactgtgaaaaatcttttaggagggttcttgagtattttaacacaggatgattctgattttcaagcatgccagagagtattggtggatcttttggtatctttgatgagttcaagaacatgttcagaagagctaacccttcttttgagaatatttctggagaaatctccttgtacaaaaattcttcttctgggtattctgaaaattattgaaagtgatactactatgagcccttcacagtatctaaccttccctttactgcacgctccaaatttaagcaacggtgtttcatcacaaaagtatcctgggattttaaacagtaaggccatgggtttattgagaagagcacgagtttcacggagcaagaaagaggctgatagagagagttttccccatcggctgctttcatcttggcacatagccccagtccacctgccgttgctggggcaaaactgctggccacacctatcagaaggtttcagtgtttccctgtggtttaatgtggagtgtatccatgaagctgagagtactacagaaaaaggaaagaagataaagaaaagaaacaaatcattaattttaccagatagcagttttgatggtacagagagcgacagaccagaaggtgcagagtacataaatcctggtgaaagactcatagaagaaggatgtattcatataatttcactgggatccaaagcgttgatgatccaagtgtgggctgatccccacaatgccactcttatctttcgtgtgtgcatggattcaaatgatgacatgaaagctgttttactagcacaggttgaatcacaggagaatattttcctcccaagcaaatggcaacatttagtactcacctacttacagcagccccaagggaaaaggaggattcatgggaaaatctccatatgggtctctggacagaggaagcctgatgttactttggattttatgcttccaagaaaaacaagtttgtcatctgatagcaataaaacattttgcatgattggccattgtttatcatcccaagaagagtttttgcagttggctggaaaatgggacctgggaaatttgcttctcttcaacggagctaaggttggttcacaagaggccttttatctgtatgcttgtggacccaaccatacatctgtaatgccatgtaagtatggcaagccagtcaatgactactccaaatatattaataaagaaattttgcgatgtgaacaaatcagagaactttttatgaccaagaaagatgtggatattggtctcttaattgaaagtctttcagttgtttatacaacttactgtcctgctcagtataccatctatgaaccagtgattagacttaaaggtcaaatgaaaacccaactctctcaaagacccttcagctcaaaagaagttcagagcatcttattagaacctcatcatctaaagaatctccaacctactgaatataaaactattcaaggcattctgcacgaaattggtggaactggcatatttgtttttctctttgccagggttgttgaactcagtagctgtgaagaaactcaagcattagcactgcgagttatactctcattaattaaatacaaccaacaaagagtacatgaattagaaaattgtaatggactttctatgattcatcaggtgttgatcaaacaaaaatgcattgttgggttttacattttgaagacccttcttgaaggatgctgtggtgaagatattatttatatgaatgagaatggagagtttaagttggatgtagactctaatgctataatccaagatgttaagctgttagaggaactattgcttgactggaagatatggagtaaagcagagcaaggtgtttgggaaactttgctagcagctctagaagtcctcatcagagcagatcaccaccagcagatgtttaatattaagcagttattgaaagctcaagtggttcatcactttctactgacttgtcaggttttgcaggaatacaaagaggggcaactcacacccatgccccgagaggtttgtagatcatttgtgaaaattatagcagaagtccttggatctcctccagatttggaattattgacaattatcttcaatttccttttagcagttcaccctcctactaatacttacgtttgtcacaatcccacgaacttctacttttctttgcacatagatggcaagatctttcaggagaaagtgcggtcaatcatgtacctgaggcattccagcagtggaggaaggtcccttatgagccctggatttatggtaataagcccatctggttttactgcttcaccatatgaaggagagaattcctctaatattattccacaacagatggccgcccatatgctgcgttctagaagcctaccagcattccctacttcttcactactaacgcaatcacaaaaactgactggaagtttgggttgtagtatcgacaggttacaaaatattgcagatacttatgttgccacccaatcaaagaaacaaaattctttggggagttccgacacactgaaaaaaggcaaagaggacgcattcatcagtagctgtgagtctgcaaaaactgtttgtgaaatggaagctgtcctctcagcccaggtctctgtcagtgatgtcccaaagggagtgctgggatttccagtggtcaaagcagatcataaacagttgggagcagaacccaggtcagaagatgacagtcctggggatgagtcctgcccacgccgacctgattacctaaagggattggcctccttccagcgaagccacagcactattgcaagccttgggctagcttttccttcacagaacggatctgcagctgttggccgttggccaagtcttgttgatagaaacactgatgattgggaaaactttgcctattctcttggttatgagccaaattacaaccgaactgcaagtgctcacagtgtaactgaagactgtttggtacctatatgctgtggattatatgaactcctaagtggggttcttcttatcctgcctgatgttttgcttgaagatgtgatggacaagcttattcaagcagatacacttttggtcctcgttaaccacccatcaccagctatacaacaaggtgttattaaactattagatgcatattttgctagagcatctaaggaacaaaaagataaatttctgaagaatcgtggattttccttgctagccaaccagttgtatcttcatcgaggaactcaagaattgttagaatgcttcatcgaaatgttctttggtcgacatattggccttgatgaagaatttgatctggaagatgtgagaaacatgggattgtttcagaagtggtctgtcattcctattctgggactaatagagacctctctatatgacaacatactcttgcataatgctcttttacttcttctccaaattttaaattcttgttctaaggtagcagatatgttgctggataatggtctactctatgtgttatgtaatacagtagcagccctgaatggattagaaaagaacattcccatgagtgaatataaattgcttgcttgtgatatacagcaacttttcatagcagttacaattcatgcttgcagttcctcaggctcacaatattttagggttattgaagaccttattgtaatgcttggatatcttcaaaatagcaaaaacaagaggacacaaaatatggctgttgcactacagcttagagttctccaggctgctatggaatttataaggaccaccgcaaatcatgactctgaaaacctcacagattcactccagtcaccttctgctccccatcatgcagtagttcaaaagcggaaaagcattgctggtcctcgaaaatttccccttgctcaaactgaatcgcttctgatgaaaatgcgttcagtggcaaatgatgagcttcatgtgatgatgcaacggagaatgagccaagagaaccctagccaagcaactgaaacggaacttgcgcagagactacagaggctcactgttttagcagtcaacaggattatttatcaagaatttaattcagacattattgacattttgagaactccagaaaatgtaactcaaagcaagacctcagttttccagaccgaaatttctgaggaaaatattcatcatgaacagtcttctgttttcaatccatttcagaaagaaatttttacatatctggtagaaggattcaaagtatctattggttcaagtaaagccagtggttccaagcagcaatggactaaaattctgtggtcttgtaaggagaccttccgaatgcagcttgggagactactagtgcatattttgtcgccagcccacgctgcacaagagagaaagcaaatttttgaaatagttcatgaaccaaatcatcaggaaatactacgagactgtctcagcccatccctacaacatggagccaagttagttttgtatttgtcagagttgatacataatcaccaaggtgaattgactgaagaagagctaggcacagcagaactgcttatgaatgctttgaagttatgtggtcacaagtgcatccctcccagtgcatcaacaaaagcagaccttattaaaatgatcaaagaggaacaaaagaaatatgaaactgaagaaggagtgaataaagctgcttggcagaaaacagttaacaataatcaacaaagtctctttcagcgtctggattcaaaatcaaaggatatatctaaaatagctgcagatatcacccaggcagtgtctctctcccaaggaaatgagagaaaaaaggtgatccagcatattagaggaatgtataaagtagatttgagtgccagcagacattggcaggaacttattcagcagctgacacatgatagagcagtatggtatgaccccatctactatccaacctcatggcagttggatccaacagaagggccaaatcgagagaggagacgtttacagagatgttatttaactattccaaataagtatctccttagggatagacagaaatcagaagatgttgtcaaaccaccactctcttacctgtttgaagacaaaactcattcttctttctcttctactgtcaaagacaaagctgcaagtgaatctataagagtgaatcgaagatgcatcagtgttgcaccatctagagagacagctggtgaattgttactaggtaaatgtggaatgtattttgtggaagataatgcttctgatacagttgaaagttcgagccttcagggagagttggaaccagcatcattttcctggacatatgaagaaattaaagaagttcacaagcgttggtggcaattgagagataatgctgtagaaatctttctaacaaatggcagaacactcctgttggcatttgataacaccaaggttcgtgatgatgtataccacaatatactcacaaataacctccctaatcttctggaatatggtaacatcaccgctctgacaaatttatggtatactgggcaaattactaattttgaatatttgactcacttaaacaaacatgctggccgatccttcaatgatctcatgcagtatcctgtgttcccatttatacttgctgactacgttagtgagacacttgacctcaatgatctgttgatatacagaaatctctctaaacctatagctgttcagtataaagaaaaagaagatcgttatgtggacacatacaagtacttggaggaagagtaccgcaaaggagccagagaagatgaccccatgcctcccgtgcagccctatcactatggctcccactattccaatagcggcactgtgcttcacttcctggtcaggatgcctcctttcactaaaatgtttttagcctatcaagatcaaagttttgacattccagacagaacttttcattctacaaatacaacttggcgactctcatcttttgaatctatgactgatgtgaaagaacttatcccagagtttttctatcttccagagttcctagttaaccgtgaaggttttgattttggtgtgcgtcagaatggtgaacgggttaatcacgtcaaccttcccccttgggcgcgtaatgatcctcgtctttttatcctcatccatcggcaggctctagagtctgactacgtgtcgcagaacatctgtcagtggattgacttggtgtttgggtataagcaaaaggggaaggcttctgttcaagcgatcaatgtttttcatcctgctacatattttggaatggatgtctctgcagttgaagatccagttcagagacgagcgctagaaaccatgataaaaacctacgggcagactccccgtcagctgttccacatggcccatgtgagcagacctggagccaagctcaatattgaaggagagcttccagctgctgtggggttgctagtgcagtttgctttcagggagacccgagaacaggtcaaagaaatcacctatccgagtcctttgtcatggataaaaggcttgaaatggggggaatacgtgggttcccccagtgctccagtacctgtggtctgcttcagccagccccacggagaaagatttggctctctccaggctctgcccaccagagcaatctgtggtttgtcacggaatttctgtcttctgatgacatatagcaaggaacaaggtgtgagaagcatgaacagtacggacattcagtggtcagccatcctgagctggggatatgctgataatattttaaggttgaagagtaaacaaagtgagcctccagtaaactttattcaaagttcacaacagtaccaggtgactagttgtgcttgggtgcctgacagttgccagctgtttactggaagcaaatgcggtgtcatcacagcctacacaaacagatttacaagcagcacgccatcagaaatagaaatggagactcaaatacatctctatggtcacacagaagagataaccagcttatttgtttgcaaaccatacagtatactgataagtgtgagcagagacggaacctgcatcatatgggatttaaacaggttatgctatgtacaaagtctggcgggacacaaaagccctgtcacagctgtctctgccagtgaaacctcaggtgatattgctactgtgtgtgattcagctggcggaggcagtgacctcagactctggacggtgaacggggatctcgttggacatgtccactgcagggagatcatctgttccgtggctttctccaaccagcctgagggagtatctatcaatgtaatcgctgggggattagaaaatggaattgtaaggttatggagcacatgggacttaaagcctgtgagagaaattacatttcccaaatcaaataagcccatcatcagccttacattttcttgtgatggccaccatttgtacacagcaaacagtgatgggaccgtgattgcctggtgtcggaaggaccagcagcgcttgaaacagccaatgttctattccttccttagcagctatgcagccgggtgaatgcgaatgaacttcacgttctccaaagcactttaactccaaactagatttgttgacttcaccagttttaggaggttgaacctaaagaaatggatgactggacaaaccatccaaataatgataaagtctattcatctgcacaaaattctgaagagtcacatgatcctaagaggaaagttctgttctattttagtgataatctggaagattgtgtcaatatgcactagccaacaagttttaagcctcgcatggtacattaaaatgatattcttaaaattttttcccaccaaggtattccaaagaaaatattaaggtctcccctttttctatgattccaaaaggaccagtagaatttaaattggttggttgattgtttatataaaacacactaaaattatattttaaaagtttactgccatgaaatactcctcccaccacacacacatgctccaaaagaggaaagaaaaaaagataatttttaggacttgataattgctttctttgagaagcaaattattcagtaggtgcctctgtaccaaatattttatggaatatctaaatactaaaataaactatgaatgaatctcaaaattaggcagtttttgccagttgctttcttagctcaaaggagaaccagaatttttttgacagccacaaacaagaatacaggtatcttggatttcagacacattctgtttcttcataaaaattttacttaaaatctgtaacgctagatattgactatccttagttgagtcactgaggtttaaacacaatggtaagtcttaaagtctgctatttacagagcattgaatctgtaccaatttgcaatagaaagccttcagtatgcaagaagtttgcatgggtattaagaacacagcctaaataaggcatttgatctaatctgcaggaagaattttcttccccaaaacagaattataaaagcttactttaaacaggaggcagaataattcttttaggaaaccatttcattctgtttctactaacctataccatctgagaattctaggaagaataataaaatctcgtgtattccacagcaaacttacataccataaagacagaattcctaaacatcttggagccgtctgtctctcccatatgatggctgtctgtatatttttacttggggtgctgctttattggctttgaaaacactgtcagataagctcagtaatatgttaccatgggataaaaatatgtatccctgcctaagaataacttgtgcatttgttatggaaatttaattcatatggtgtttacagtactacttttgtaacttccagactttctaaaacattctgcttaaaaaccatataaaatataattccaaagtctctgctgtcaagatagattcgagagaaagcacgtggccatgtatgctttaaccttaaactgcatacacatgtagtgatacctaggctgcatttagatcaccgtgtgctcaggccaggtgtgaatcctgaggtccatggaggtgcagagatgagattactcctattcacgttgaagtgatttgctttgttaacaaaaaattgcagctattgtctagctttcatttttttactgagaactttaaattagtcccctattagaatagggttgctactcatctttttttaaaaaccgaatttcatcatttatctaaagagaaaatatgcagaataactggtcttgttaagagtgcaatattatatttttatgtaaaaataaaaattaatttggggggattatttattcagcatgaaacctaatatgtatatgtttgaaatacttcataatgtgcatgttgtagcaaacatttctgtaaattatcacaagctctgttacctttatatacactgcctcttcaatttggaaataaatttcataaaaatagatgtaaacaaaaaaa

SEQ ID NO: 2 - protein, 3801 aa, Homo sapiens
MSTDSNSLAREFLTDVNRLCNAVVQRVEAREEEEEETHMATLGQYLVHGRGFLLLTKLNSIIDQALTCREELLTLLLSLLPLVWKIPVQEEKATDFNLPLSADIILTKEKNSSSQRSTQEKLHLEGSALSSQVSAKVNVFRKSRRQRKITHRYSVRDARKTQLSTSDSEANSDEKGIAMNKHRRPHLLHHFLTSFPKQDHPKAKLDRLATKEQTPPDAMALENSREIIPRQGSNTDILSEPAALSVISNMNNSPFDLCHVLLSLLEKVCKFDVTLNHNSPLAASVVPTLTEFLAGFGDCCSLSDNLESRVVSAGWTEEPVALIQRMLFRTVLHLLSVDVSTAEMMPENLRKNLTELLRAALKIRICLEKQPDPFAPRQKKTLQEVQEDFVFSKYRHRALLLPELLEGVLQILICCLQSAASNPFYFSQAMDLVQEFIQHHGFNLFETAVLQMEWLVLRDGVPPEASEHLKALINSVMKIMSTVKKVKSEQLHHSMCTRKRHRRCEYSHFMHHHRDLSGLLVSAFKNQVSKNPFEETADGDVYYPERCCCIAVCAHQCLRLLQQASLSSTCVQILSGVHNIGICCCMDPKSVIIPLLHAFKLPALKNFQQHILNILNKLILDQLGGAEISPKIKKAACNICTVDSDQLAQLEETLQGNLCDAELSSSLSSPSYRFQGILPSSGSEDLLWKWDALKAYQNFVFEEDRLHSIQIANHICNLIQKGNIVVQWKLYNYIFNPVLQRGVELAHHCQHLSVTSAQSHVCSHHNQCLPQDVLQIYVKTLPILLKSRVIRDLFLSCNGVSQIIELNCLNGIRSHSLKAFETLIISLGEQQKDASVPDIDGIDIEQKELSSVHVGTSFHHQQAYSDSPQSLSKFYAGLKEAYPKRRKTVNQDVHINTINLFLCVAFLCVSKEAESDRESANDSEDTSGYDSTASEPLSHMLPCISLESLVLPSPEHMHQAADIWSMCRWIYMLSSVFQKQFYRLGGFRVCHKLIFMIIQKLFRSHKEEQGKKEGDTSVNENQDLNRISQPKRTMKEDLLSLAIKSDPIPSELGSLKKSADSLGKLELQHISSINVEEVSATEAAPEEAKLFTSQESETSLQSIRLLEALLAICLHGARTSQQKMELELPNQNLSVESILFEMRDHLSQSKVIETQLAKPLFDALLRVALGNYSADFEHNDAMTEKSHQSAEELSSQPGDFSEEAEDSQCCSFKLLVEEEGYEADSESNPEDGETQDDGVDLKSETEGFSASSSPNDLLENLTQGEIIYPEICMLELNLLSASKAKLDVLAHVFESFLKIIRQKEKNVFLLMQQGTVKNLLGGFLSILTQDDSDFQACQRVLVDLLVSLMSSRTCSEELTLLLRIFLEKSPCTKILLLGILKIIESDTTMSPSQYLTFPLLHAPNLSNGVSSQKYPGILNSKAMGLLRRARVSRSKKEADRESFPHRLLSSWHIAPVHLPLLGQNCWPHLSEGFSVSLWFNVECIHEAESTTEKGKKIKKRNKSLILPDSSFDGTESDRPEGAEYINPGERLIEEGCIHIISLGSKALMIQVWADPHNATLIFRVCMDSNDDMKAVLLAQVESQENIFLPSKWQHLVLTYLQQPQGKRRIHGKISIWVSGQRKPDVTLDFMLPRKTSLSSDSNKTFCMIGHCLSSQEEFLQLAGKWDLGNLLLFNGAKVGSQEAFYLYACGPNHTSVMPCKYGKPVNDYSKYINKEILRCEQIRELFMTKKDVDIGLLIESLSVVYTTYCPAQYTIYEPVIRLKGQMKTQLSQRPFSSKEVQSILLEPHHLKNLQPTEYKTIQGILHEIGGTGIFVFLFARVVELSSCEETQALALRVILSLIKYNQQRVHELENCNGLSMIHQVLIKQKCIVGFYILKTLLEGCCGEDIIYMNENGEFKLDVDSNAIIQDVKLLEELLLDWKIWSKAEQGVWETLLAALEVLIRADHHQQMFNIKQLLKAQVVHHFLLTCQVLQEYKEGQLTPMPREVCRSFVKIIAEVLGSPPDLELLTIIFNFLLAVHPPTNTYVCHNPTNFYFSLHIDGKIFQEKVRSIMYLRHSSSGGRSLMSPGFMVISPSGFTASPYEGENSSNIIPQQMAAHMLRSRSLPAFPTSSLLTQSQKLTGSLGCSIDRLQNIADTYVATQSKKQNSLGSSDTLKKGKEDAFISSCESAKTVCEMEAVLSAQVSVSDVPKGVLGFPVVKADHKQLGAEPRSEDDSPGDESCPRRPDYLKGLASFQRSHSTIASLGLAFPSQNGSAAVGRWPSLVDRNTDDWENFAYSLGYEPNYNRTASAHSVTEDCLVPICCGLYELLSGVLLILPDVLLEDVMDKLIQADTLLVLVNHPSPAIQQGVIKLLDAYFARASKEQKDKFLKNRGFSLLANQLYLHRGTQELLECFIEMFFGRHIGLDEEFDLEDVRNMGLFQKWSVIPILGLIETSLYDNILLHNALLLLLQILNSCSKVADMLLDNGLLYVLCNTVAALNGLEKNIPMSEYKLLACDIQQLFIAVTIHACSSSGSQYFRVIEDLIVMLGYLQNSKNKRTQNMAVALQLRVLQAAMEFIRTTANHDSENLTDSLQSPSAPHHAVVQKRKSIAGPRKFPLAQTESLLMKMRSVANDELHVMMQRRMSQENPSQATETELAQRLQRLTVLAVNRIIYQEFNSDIIDILRTPENVTQSKTSVFQTEISEENIHHEQSSVFNPFQKEIFTYLVEGFKVSIGSSKASGSKQQWTKILWSCKETFRMQLGRLLVHILSPAHAAQERKQIFEIVHEPNHQEILRDCLSPSLQHGAKLVLYLSELIHNHQGELTEEELGTAELLMNALKLCGHKCIPPSASTKADLIKMIKEEQKKYETEEGVNKAAWQKTVNNNQQSLFQRLDSKSKDISKIAADITQAVSLSQGNERKKVIQHIRGMYKVDLSASRHWQELIQQLTHDRAVWYDPIYYPTSWQLDPTEGPNRERRRLQRCYLTIPNKYLLRDRQKSEDVVKPPLSYLFEDKTHSSFSSTVKDKAASESIRVNRRCISVAPSRETAGELLLGKCGMYFVEDNASDTVESSSLQGELEPASFSWTYEEIKEVHKRWWQLRDNAVEIFLTNGRTLLLAFDNTKVRDDVYHNILTNNLPNLLEYGNITALTNLWYTGQITNFEYLTHLNKHAGRSFNDLMQYPVFPFILADYVSETLDLNDLLIYRNLSKPIAVQYKEKEDRYVDTYKYLEEEYRKGAREDDPMPPVQPYHYGSHYSNSGTVLHFLVRMPPFTKMFLAYQDQSFDIPDRTFHSTNTTWRLSSFESMTDVKELIPEFFYLPEFLVNREGFDFGVRQNGERVNHVNLPPWARNDPRLFILIHRQALESDYVSQNICQWIDLVFGYKQKGKASVQAINVFHPATYFGMDVSAVEDPVQRRALETMIKTYGQTPRQLFHMAHVSRPGAKLNIEGELPAAVGLLVQFAFRETREQVKEITYPSPLSWIKGLKWGEYVGSPSAPVPVVCFSQPHGERFGSLQALPTRAICGLSRNFCLLMTYSKEQGVRSMNSTDIQWSAILSWGYADNILRLKSKQSEPPVNFIQSSQQYQVTSCAWVPDSCQLFTGSKCGVITAYTNRFTSSTPSEIEMETQIHLYGHTEEITSLFVCKPYSILISVSRDGTCIIWDLNRLCYVQSLAGHKSPVTAVSASETSGDIATVCDSAGGGSDLRLWTVNGDLVGHVHCREIICSVAFSNQPEGVSINVIAGGLENGIVRLWSTWDLKPVREITFPKSNKPIISLTFSCDGHHLYTANSDGTVIAWCRKDQQRLKQPMFYSFLSSYAAG

(19) United States
(12) Patent Application Publication
(10) Pub. No.: US 2021/0238266 A1
(43) Pub. Date: Aug. 5, 2021

(54) COMPOSITIONS AND METHODS FOR ANTI-LYST IMMUNOMODULATION

(71) Applicant: Research Institute at Nationwide Children's Hospital, Columbus, OH (US)

(72) Inventors: Christopher Breuer, Columbus, OH (US); Narutoshi Hibino, Columbus, OH (US); Vidu Garg, Columbus, OH (US); Cameron Best, Columbus, OH (US)

(21) Appl. No.: 17/098,076

(22) Filed: Nov. 13, 2020

Related U.S. Application Data
(63) Continuation of application No. 15/308,445, filed on Nov. 2, 2016.
(60) Provisional application No. 61/987,910, filed on May 2, 2014.

(51) Int. Cl. C07K 16/18; A61L 27/54; A61L 31/16; A61K 31/713; A61L 27/36; A61K 9/00; A61K 31/7105; A61L 29/16; A61L 27/50; A61K 45/06

(52) U.S. Cl. CPC C07K 16/18; A61L 27/54; A61L 31/16; A61K 31/713; A61L 27/36; A61K 9/0019; A61K 2039/505; A61L 29/16; A61L 27/507; A61K 45/06; A61L 2300/256; A61L 2300/258; A61L 2300/42; A61K 31/7105

(57) ABSTRACT

Excessive or repeated activation of inflammatory and pro-coagulant mechanisms at the site of tissue injury contributes to the development scar tissue that can lead to intimal hyperplasia and fibrotic disease. It has been established that inhibition of the LYST protein is associated with reduced inflammatory responses and reduced platelet activation at the site of tissue damage. Compositions and methods for inhibition of the expression and function of the LYST protein are described. The compositions and methods can be useful for the modulation of immune processes that contribute to formation of neointima and fibroproliferative disorders by altering macrophage, platelet and natural killer cell function to create a pro-regenerative immune response.

### CROSS-REFERENCE TO RELATED APPLICATIONS

This application is a continuation of U.S. application Ser. No. 15/308,445, filed Nov. 2, 2016, which is a National Phase application under 35 U.S.C. § 371 of PCT/US2015/029014, filed May 4, 2015, which claims priority to and benefit of U.S. Provisional Application No. 61/987,910 entitled “Compositions and Methods For Anti-Lyst Immunomodulation” filed May 2, 2014, the contents of which are incorporated by reference in their entirety.

### STATEMENT REGARDING FEDERALLY SPONSORED RESEARCH OR DEVELOPMENT

This invention was made with Government Support under grant No. HL098228 awarded by the National Institutes of Health. The Government has certain rights in the invention.

### REFERENCE TO SEQUENCE LISTING

The Sequence Listing submitted January 27, 2021, as a text file named “NWCH_2014-002_CON_ST25.txt,” created on Dec. 10, 2020, and having a size of 51,818 bytes is hereby incorporated by reference pursuant to 37 C.F.R. § 1.52(e)(5).

### FIELD OF THE INVENTION

The field of the invention is generally related to compositions, devices, and methods for enhancing vascular tissue regeneration, as well as reducing morbidity and mortality associated with neointimal hyperplasia and fibroproliferative disease.

### BACKGROUND OF THE INVENTION

Tissue repair is a complex, step-wise process that facilitates the ordered removal and replacement of dead or damaged cells in response to stimulatory cytokine signaling from local pro-inflammatory cells at the site of injury (Wynn and Baron, Semin Liver Dis., 30(3):245-257 (2010); Wynn, Clin. Invest. 117:524-529 (2007)). However, when tissue-repair responses are inappropriate or persist for an extended period, for example, in response to chronic disease or repeated injury, the normal regenerative mechanisms can give rise to pathological states resulting from excessive neotissue growth and accumulation of extracellular matrix (ECM) components at the site of injury.

Excessive or repeated activation of inflammatory and pro-coagulant mechanisms at the site of vascular injury is thought to contribute significantly to the development of intimal hyperplasia. Under pathological conditions, vascular injury causes denudation of the endothelial layer and triggers a series of acute and chronic inflammatory responses characterized by production of various growth factors and inflammatory cytokines (Murakami, et al., Am J Physiol Lung Cell Mol Physiol., 272:L197-L202 (1997); Cotran, et al., J Am Soc Nephrol., 1:225-235 (1990)). Intimal hyperplasia occurring after surgical procedures such as angioplasty and stenting, surgical bypasses or endarterectomy for coronary and/or peripheral arterial disease can cause restenosis of blood vessels in up to 80% of patients (Seedial, et al., J Vasc Surg., 57(5):1403-1414 (2013); Glagov, Circulation, 89:2888-2891 (1994)). It has been shown that neointima formation can be accelerated by the presence of foreign material such as prostheses within blood vessels, and uncontrolled neointimal hyperplasia is a major obstacle limiting the long-term clinical efficiency of cardiovascular intervention (Frank, et al., Curr. Opin. Lipidol., 15:523 (2004)). In addition, neointima formation is associated with the development and progression of several proliferative cardiovascular diseases, including atherosclerosis, hypertension, and diabetic vascular complications.

Regeneration of the endothelial layer inhibits the development of neointima and facilitates vascular repair (Bauters, et al., Prog. Cardiovasc. Dis., 40:107-116 (1997); Kinlay, et al., Curr. Opin. Lipidol., 12:383 (2001)). However, currently available anti-neointimal drugs indiscriminately block the proliferation of both endothelial cells (EC) and smooth muscle cells (SMC) resulting in impaired re-endothelialization and prolonging the wound healing process. There remains a need to develop an anti-proliferation strategy that is SMC-sensitive.

Unregulated inflammatory responses to repeated tissue damage can also give rise to pathological fibrosis of tissue and organs such as the skin, liver, heart and lung (Huang and Ogawa, Connect Tissue Res., 53(3):187-196 (2012)). Chronic damage and inflammation of liver tissue leads to the accumulation of ECM proteins that cause excessive scar formation and distort the hepatic architecture, which in turn leads to cirrhosis and liver failure (Battler and Brenner, J Clin Invest., 115:209-218 (2005)). Likewise, excessive deposition of fibrotic tissue within the lung gives rise to progressive scarring and irreversible destruction of lung architecture that ultimately leads to organ malfunction, disruption of gas exchange, and death from respiratory failure.

Activated platelets increase leukocyte adhesion to the damaged endothelium and promote leukocyte activation through deposition of chemokines on the endothelium. This enables leukocytes to firmly attach to the vessel wall and transmigrate into the subendothelial tissue. However, the platelet-derived chemokines are also known to play a role in neointimal proliferation, restenosis after arterial injury and atherosclerosis (Chandrasekar, et al., J Am College Cardiology, Vol 35, No. 3, pp. 555-562 (2000)). Certain patient groups having abnormal platelet function, including those with renal disease, diabetes mellitus and hyperlipidemia, are also at increased risk for restenosis.

Fibroproliferative disorders are a leading cause of morbidity and mortality in the United States (Bitterman and Henke, Chest, 99(3):s81-s84 (1991)). In particular, hepatic and pulmonary fibrotic diseases are typically refractory to treatment and carry a high mortality rate. Established liver cirrhosis has a 10-year mortality rate of 34-66%, and is responsible for approximately 27,000 deaths each year in the United States alone. The prevalence of idiopathic pulmonary fibrosis (IPF) is estimated at between 14 and 42.7 per 100,000, and this figure has been rising decade after decade for the last 30 years (Olson, et al., Am J Respir Crit Care Med. 176:277-2843 (2007); Gribbin, et al., Thorax, 61:980-985 (2006)). Currently, the most effective treatment for hepatic or pulmonary fibrosis is organ transplantation.

Therefore, it is an object of the invention to provide compositions, devices, grafts, and methods of use thereof for modulating immune processes to reduce or prevent neointima formation, stenosis, restenosis or a combination thereof in a subject.

It is also an object of the invention to provide compositions, devices, grafts, and methods of use thereof to reduce or prevent inappropriate or deleterious platelet activation in a subject.

It is also an object of the invention to provide compositions, methods, and devices for reducing or preventing the immune response to an implantable prosthesis in a subject.

It is a further object of the invention to provide compositions, methods, and devices to promote healing and inhibit scar formation, adhesions and fibroproliferative diseases in a subject.

### SUMMARY OF THE INVENTION

It has been established that inhibition of the lysosomal trafficking regulator (“LYST”) protein is associated with reduced inflammatory responses at the site of tissue damage. Compositions and methods for inhibition of the expression and function of the LYST protein are described.

The compositions and methods can be useful for the modulation of immune processes that contribute to formation of neointima and fibroproliferative disorders by altering macrophage, platelet and natural killer cell function to create a pro-regenerative immune response.

Compositions including one or more LYST inhibitors in an amount effective to reduce or prevent the formation of scar tissue in a subject are disclosed. Compositions including one or more LYST inhibitors in an amount effective to reduce or prevent the activation of platelets in a subject are also disclosed.

Typically the compositions include a physiologically acceptable carrier. In preferred embodiments, the amount of one or more LYST inhibitors does not prevent vascular neotissue formation in the subject. Typically, the compositions include one or more LYST inhibitors in an amount between 0.1-1000 mg/kg body weight of a human.

In one embodiment one or more LYST inhibitors is an antibody.

In another embodiment one or more LYST inhibitors is a functional nucleic acid. Exemplary functional nucleic acids include an antisense molecule, siRNA, miRNA, aptamers, ribozymes, triplex forming molecules, RNAi, and external guide sequences. The one or more functional nucleic acids can be expressed from an expression vector.

In some embodiments the compositions of one or more LYST inhibitors include a delivery vehicle. Exemplary delivery vehicles include nanoparticles, microparticles, micelles, synthetic lipoprotein particles, liposomes, and carbon nanotubes. The disclosed compositions can also include one or more additional therapeutic agents. Vascular grafts and medical devices including one or more LYST inhibitors are disclosed. In some embodiments the composition of one or more LYST inhibitors is coated onto or incorporated into the graft or device. Exemplary medical devices include stents, implants, needles, cannulas, catheters, shunts, balloons, and valves. The medical device can be a stent, such as a drug eluting stent that elutes the composition of one or more LYST inhibitors. Exemplary vascular grafts include autologous, preserved autologous, allogeneic, xenogenic or synthetic grafts.

Methods for reducing or preventing the formation of scar tissue in a subject, including administering to a subject in need thereof a composition including one or more LYST inhibitors are also provided. Methods for reducing or preventing the development of venous thrombosis or arterial thrombosis in a subject include administering to a subject in need thereof a composition including one or more LYST inhibitors. In some embodiments the subject is at risk of developing, or has developed, restenosis or other vascular proliferation disorders. In further embodiments the subject has undergone, is undergoing, or will undergo vascular trauma, angioplasty, vascular surgery, or transplantation arteriopathy. The methods can promote healing, reduce or prevent the development of hypertrophic scarring, keloids, or adhesions, reduce or prevent fibrosis of the liver, fibrosis of the lungs, fibrosis of the heart or fibrosis of the kidneys, reduces or prevents neointima formation, stenosis or restenosis, or any combination thereof in the subject relative to an untreated control subject. The methods can promote integration but block encapsulation of one or more prosthetic devices such as pacemakers, nerve stimulators, pacemaker leads, replacement heart valves and artificial joints or components thereof. The methods can be effective to treat or prevent neointima formation at a site of implantation of a vascular implant, a site of vascular injury, or a site of surgery in the subject, relative to an untreated control subject. The methods can be effective to reduce or prevent the expression of platelet derived growth factor, transforming growth factor beta, or any combination thereof in a subject relative to an untreated control subject.

### BRIEF DESCRIPTION OF THE DRAWINGS

FIG. 1 is a histogram showing the diameter (mm) of the conduit within biodegradable conduit grafts two weeks after implant into beige mice (bg; black graph) and wild type mice (WT; grey graph). N=10 and N=25 for beige mice and WT mice, respectively.

FIG. 2 is a histogram showing the number of macrophage cells (cell/HPF) within biodegradable conduit grafts explanted from beige mice (Bg; grey graph) and wild type mice (WT; black graph), respectively, two weeks after implant. P=0.0048.

FIG. 3 is a schematic showing the difference between the nucleotide sequences (at top) of the wild type (WT) and Beige (Bg) mice in the LYST gene on exon 52. Corresponding differences between the amino acid sequences of the LYST protein of WT and Bg mice are also shown in an amino acid sequence alignment (middle). The amino acid sequence alignment (at bottom) shows conservation amongst the amino acid sequences of LYST polypeptides from human, mouse and rat. The position of the deletion within the LYST of Beige (Bg) mice is indicated by *. Nucleic acids and amino acids are represented using the respective standard single-letter codes.

FIG. 4 is a histogram showing the relative level of mRNA corresponding to the LYST gene product, amplified using each of 5 different oligonucleotide primers (LYST-1, LYST-3, LYST-4, LYST-5 and LYST-6), in cells obtained from wild-type mice (WT-1(1); WT-2 (2)), Beige mice (Beige-1 (3); Beige-2 (4)) or a standard “Raw” cell line (5), respectively.

FIG. 5 is a histogram showing the incidence of stenosis (%) in C57BL/6 (wild type) mice and C57BL/6 Lysttmlb knockout mice, respectively, (n=5) after 2-week implantation . ***p=0.0001.

FIG. 6 is a histogram showing the amount of platelet derived growth factor (PDGF) secreted from PRP (μg/ml) in resting (control) and thrombin activated C57BL/6 (wild type) mice, as well as C57BL/6 Beige (Bg) mice, respectively. Total concentrations of secreted PDGF from each group (n=7) were compared to resting, non-activated controls (n=5).

### DETAILED DESCRIPTION OF THE INVENTION

### I. Definitions

The term “stenosis” refers to an abnormal narrowing in a blood vessel that occurs following an injury to the vessel wall (endothelium). In some embodiments, stenosis involves a reduction in the circumference of a lumen of 50% or more. The term “restenosis” refers to stenosis at a previously stenotic site or narrowing of the lumen of a blood vessel or synthetic graft following an interventional procedure. Restenosis, as used herein, encompasses occlusion. Exemplary injuries that result in stenosis or restenosis include trauma to an atherosclerotic lesion (as seen with angioplasty or stent), a resection of a lesion (as seen with endarterectomy), an external trauma (e.g., a cross-clamping injury), or a surgical anastomosis.

The term “neointimal stenosis” refers to abnormal narrowing in a blood vessel resulting from neointimal formation.

The term “neointima” refers to a renewed or thickened layer of intima (inner lining) formed in a blood vessel in response to signals from injured endothelial cells.

The terms “scar tissue” and “scarring” refer to the fibrous tissue that is produced to replace damaged tissue following injury.

The terms “fibroproliferative disorders”, “FPD” and “fibrotic diseases” are used interchangeably and include benign and malignant diseases or conditions that result from the abnormal and excessive deposition of connective tissue.

The term “Platelet Activation” is the step-wise physiological process that gives rise to adherence and aggregation of circulating platelets in response to tissue injury, such as damage or interruption of the endothelium. Platelet activation gives rise to expression and secretion of chemotactic agents such as platelet derived growth factor (PDGF) and transforming growth factor beta (TGFβ).

“Pharmaceutically acceptable carrier” encompasses any of the standard pharmaceutical carriers, such as a phosphate buffered saline solution, water and emulsions such as an oil/water or water/oil emulsion, and various types of wetting agents.

“Inhibit” or other forms of the word such as “inhibiting” or “inhibition” means to hinder or restrain a particular characteristic. It is understood that this is typically in relation to some standard or expected value, i.e., it is relative, but that it is not always necessary for the standard or relative value to be referred to. For example, “inhibits LYST” means hindering, interfering with or restraining the activity of the LYST gene relative to a standard or a control. “Inhibits LYST” can also mean to hinder or restrain the synthesis, expression or function of the LYST protein relative to a standard or control.

“Treatment” or “treating” means to administer a composition to a subject or a system with an undesired condition (e.g., restenosis or a fibroproliferative disorder). The condition can include a disease. “Prevention” or “preventing” means to administer a composition to a subject or a system at risk for the condition. The condition can be a predisposition to a disease. The effect of the administration of the composition to the subject (either treating and/or preventing) can be, but is not limited to, the cessation of a particular symptom of a condition, a reduction or prevention of the symptoms of a condition, a reduction in the severity of the condition, the complete ablation of the condition, a stabilization or delay of the development or progression of a particular event or characteristic, or minimization of the chances that a particular event or characteristic will occur.

As used herein, the term “antibody” is used in the broadest sense unless clearly indicated otherwise. Therefore, an “antibody” can be naturally occurring or man-made, such as monoclonal antibodies produced by conventional hybridoma technology. Antibodies include monoclonal and polyclonal antibodies as well as fragments containing the antigen-binding domain and/or one or more complementarity determining regions of these antibodies. “Antibody” refers to any form of antibody or antigen binding fragment thereof and includes monoclonal antibodies (including full length monoclonal antibodies), polyclonal antibodies, multi-specific antibodies (e.g., bi-specific antibodies), and antibody fragments.

### II. Compositions

It has been established that the processes of tissue repair can be mediated by manipulation of the expression and function of the LYST gene. Specifically, the loss of function of the LYST gene product gives rise to abnormal function in certain immune cells, resulting in immune dis-function and an associated reduction in proliferative disorders Immune cells that can be influenced by modulation of the LYST gene or LYST protein include innate immune cells, macrophages and platelets.

Innate Immune Cells

Innate immune processes including inflammation, removal of damaged cells and debris, as well as the development of neotissue and other wound repair mechanisms are an important component of the physiological responses to injury. Tissue repair has four distinct stages, including: a) clotting/coagulation; b) inflammation; c) fibroblast migration/proliferation; and d) a final remodeling phase where normal tissue architecture is restored. In the earliest stages after tissue damage, epithelial cells and/or endothelial cells release inflammatory mediators that initiate an antifibrinolytic-coagulation cascade that triggers clotting and development of a provisional extracellular matrix (ECM). Aggregation and subsequent degranulation of platelets promotes blood vessel dilation and increased permeability, allowing efficient recruitment of inflammatory cells such as neutrophils, macrophages, lymphocytes, and eosinophils to the damaged tissue. Neutrophils are the most abundant inflammatory cell at the earliest stages of wound healing, but are quickly replaced by macrophages after neutrophil degranulation. Activated macrophages and neutrophils debride the wound, eliminate any invading organisms and produce a variety of cytokines and chemokines that amplify the inflammatory response as well as trigger fibroblast proliferation and recruitment. Upon activation, fibroblasts transform into myofibroblasts that secrete α-smooth muscle actin and ECM components. Finally, in the remodeling phase epithelial/endothelial cells divide and migrate over the temporary matrix to regenerate the damaged tissue. Thus, healing and neotissue generation is a finely regulated process that balances the need to regenerate tissue and thicken blood vessel walls, without excessive thickening and stenosis or fibrosis.

Macrophages

It has been shown that the presence of circulating monocytes and infiltrating macrophages is critical for wound healing and neotis sue development (Arras, et al., J Clin Invest, 101(1): 40-50 (1998)). However, the extent of macrophage infiltration at a site of tissue damage has also been correlated with proliferative dysregulation and neointima formation (Hibino, et al., FASEB J. 25(12):4253-63 (2011)). Further, numerous studies have indicated that macrophages and fibroblasts are the main effector cells involved in the pathogenesis of fibrosis (reviewed in Wynn, Nat Rev Immunol. 4(8):583-94 (2004)).

Following vascular damage, inflammatory monocyte cells (CD16-hi, CD64-hi and CD14-hi in humans; CD115+, CD11b+ and Ly6c-hi in mice) are recruited to the damaged tissue and differentiate into activated macrophages (Emrl-hi in humans; F4/80-hi in mice) upon exposure to local growth factors, pro-inflammatory cytokines and microbial compounds (Geissmann et al., Science 327: 656-661 (2010)). Excessive macrophage infiltration results in stenosis, whilst complete inhibition of macrophage infiltration prevents neotissue formation (Hibino, et al., FASEB J. 25(12):4253-63 (2011).

Two distinct states of polarized activation for macrophages have been defined: the classically activated (M1) macrophage phenotype and the alternatively activated (M2) macrophage phenotype (Gordon and Taylor, Nat. Rev. Immunol. 5: 953-964 (2005); Mantovani et al., Trends Immunol. 23: 549-555 (2002)). The role of the classically activated (M1) macrophage is an effector cell in TH1 cellular immune responses, whereas the alternatively activated (M2) macrophage appears to be involved in immunosuppression and wound healing/tissue repair. M1 and M2 macrophages have distinct chemokine and chemokine receptor profiles, with M1 secreting the TH1 cell-attracting chemokines CXCL9 and CXCL10, and with M2 macrophages expressing chemokines CCL17, CCL22 and CCL24.

The presence of M2 macrophages has been associated with neo-intima development and stenosis (Hibino, et al., FASEB J. 25(12):4253-63 (2011)). The correlation between the extent of macrophage infiltration, neotissue formation and stenosis at certain time points following tissue graft implantation provides means to prevent stenosis through modulation of macrophage activity.

Further, macrophages are typically located close to collagen-producing myofibroblast cells, and it has been shown that monocyte-derived macrophages critically perpetuate inflammatory responses after injury as a prerequisite for fibrosis (Wynn and Barron, Semin Liver Dis., 30(3):245-257 (2010)). Macrophages produce pro-fibrotic mediators that activate fibroblasts, including platelet-derived growth factor (PDGF), a potent chemotactic agent, and transforming growth factor beta (TGF-B). Specifically, a marked increase of the non-classical M2 (CD14+, CD16+) subset of macrophages has been correlated with pro-inflammatory cytokines and clinical progression in patients suffering from chronic liver disease. During fibrosis progression, monocyte-derived macrophages release cytokines perpetuating chronic inflammation as well as directly activate hepatic stellate cells (HSCs), resulting in their proliferation and trans-differentiation into collagen-producing myofibroblasts (Zimmermann, et al., PLOS One, 5(6):e11049 (2010)).

Platelets

Aggregated platelets assist the repair of blood vessels by secreting chemicals that attract fibroblasts from surrounding connective tissue into the wounded area to heal the wound or, in the case of dysregulated inflammatory responses, form scar tissue. In response to tissue injury, platelets become activated and release a multitude of growth factors which stimulate the deposition of extracellular matrix, such as platelet-derived growth factor (PDGF), a potent chemotactic agent, as well as transforming growth factor beta (TGF-β). Both of these growth factors have been shown to play a significant role in the repair and regeneration of connective tissues. PDGF functions as a primary mitogen and chemo-attractant which significantly augments the influx of fibroblasts and inflammatory cells, as well as stimulating cell proliferation and gene expression. PDGF enables leukocytes to firmly attach to the vessel wall and finally to transmigrate into the subendothelial tissue. However, the platelet-derived chemokines are also known to induce smooth muscle cell (SMC) proliferation and play a role in neointimal proliferation and organ fibrosis (Chandrasekar, et al., J Am College Cardiology, Vol 35, No. 3, pp. 555-562 (2000)). Increased expression of PDGF and its receptors is associated with scleroderma lung and skin tissue. Specifically, there is evidence for an autocrine PDGF-receptor mediated signaling loop in scleroderma lung and skin fibroblasts, implicating both TGF-β and PDGF pathways in chronic fibrosis in scleroderma (Trojanowska, Rheumatology; 47:v2-v4 (2008)). In addition, deregulation of PDGF signaling is associated with cardiovascular indications such as pulmonary hypertension, and atherosclerosis.

Media layer smooth muscle cell (SMC) proliferation and migration in response to injury-induced PDGF are essential events contributing to neointimal thickening (Fingerle, et al., Proc Natl Acad Sci., 86:8412 (1989); Clowes, et al., Circ. Res., 56:139-145 (1985)) which eventually leads to blood vessel narrowing and stenosis.

Other healing-associated growth factors released by platelets include basic fibroblast growth factor, insulin-like growth factor 1, platelet-derived epidermal growth factor, and vascular endothelial growth factor.

Thus, inhibitors of the LYST gene and LYST protein can create a pro-regenerative immune environment that enhances wound healing and prevents proliferative disorders such as intimal hyperplasia, development of excessive scar tissue and fibrotic disease. Modulation of LYST can also modulate platelet activity and function. Thus, inhibitors of the LYST gene and/or LYST protein can be used to reduce or prevent the biological functions of platelets, such as platelet aggregation and the production/expression of platelet derived growth factor (PDGF).

Compositions for promoting tissue regeneration and preventing or reducing diseases characterized by the formation of excessive scar tissue by blockade of expression and/or function of the LYST protein are disclosed.

A. LYST

The lysosomal trafficking regulator (LYST) gene product is an ubiquitous protein associated with the transport of intracellular material to the lysosome. It has been established that mutations which reduce or inhibit the function of the LYST protein interfere with the normal functions of macrophages and also impact the biological activities of natural killer (NK) cells and platelets. It is believed that modulation of the LYST protein provides a means to modulate immune processes that give rise to vascular proliferative disorders, neointima formation, fibrosis and excessive scarring.

1. The LYST Gene

The human LYST gene is located at chromosome 1, (segment 1q42.1-q42.2; base pairs 235,661,030 to 235,883,707) (Barrat, et al., Am. J. Hum. Genet, 59:625-632 (1996)). Nucleic acid sequences for the LYST gene product are known in the art. See, for example, NCBI Reference Sequence: NM_000081.3, Homo sapiens lysosomal trafficking regulator (LYST), transcript variant 1, mRNA, which provides the nucleic acid sequence:

| (SEQ ID NO: 1) | |
|---|---|
| 1 | GTGCGCGGCG GCGGCGGCAG CGGCGTCGGC TCGGGGTTCT CCGGGAGAGG GGGAGTGCGC |
| 61 | GGCGGCCGCA GCTGCCACAA ACCAGGTGAA GCTTTGTTCT AAGAATATTT GTTTCATCTA |
| 121 | GTTTATGAGT CCAAATGATA TAGACTGTAA ATGTCACAGC AGTGGTGAAA GACTGCTCGG |
| 181 | TCATGAGCAC CGACAGTAAC TCACTGGCAC GTGAATTTCT GACCGATGTC AACCGGCTTT |
| 241 | GCAATGCAGT GGTCCAGAGG GTGGAGGCCA GGGAGGAAGA AGAGGAGGAG ACGCACATGG |
| 301 | CAACCCTTGG ACAGTACCTT GTCCATGGTC GAGGATTTCT ATTACTTACC AAGCTAAATT |
| 361 | CTATAATTGA TCAGGCATTG ACATGTAGAG AAGAACTCCT GACTCTTCTT CTGTCTCTCC |
| 421 | TTCCACTGGT ATGGAAGATA CCTGTCCAAG AAGAAAAGGC AACAGATTTT AACCTACCGC |
| 481 | TCTCAGCAGA TATAATCCTG ACCAAAGAAA AGAACTCAAG TTCACAAAGA TCCACTCAGG |
| 541 | AAAAATTACA TTTAGAAGGA AGTGCCCTGT CTAGTCAGGT TTCTGCAAAA GTAAATGTTT |
| 601 | TTCGAAAAAG CAGACGACAG CGTAAAATTA CCCATCGCTA TTCTGTAAGA GATGCAAGAA |
| 661 | AGACACAGCT CTCCACCTCA GATTCAGAAG CCAATTCAGA TGAAAAAGGC ATAGCAATGA |
| 721 | ATAAGCATAG AAGGCCCCAT CTGCTGCATC ATTTTTTAAC ATCGTTTCCT AAACAAGACC |
| 781 | ACCCCAAAGC TAAACTTGAC CGCTTAGCAA CCAAAGAACA GACTCCTCCA GATGCTATGG |
| 841 | CTTTGGAAAA TTCCAGAGAG ATTATTCCAA GACAGGGGTC AAACACTGAC ATTTTAAGTG |
| 901 | AGCCAGCTGC CTTGTCTGTT ATCAGTAACA TGAACAATTC TCCATTTGAC TTATGTCATG |
| 961 | TTTTGTTATC TTTATTAGAA AAAGTTTGTA AGTTTGACGT TACCTTGAAT CATAATTCTC |
| 1021 | CTTTAGCAGC CAGTGTAGTG CCCACACTAA CTGAATTCCT AGCAGGCTTT GGGGACTGCT |
| 1081 | GCAGTCTGAG CGACAACTTG GAGAGTCGAG TAGTTTCTGC AGGTTGGACC GAAGAACCGG |
| 1141 | TGGCTTTGAT TCAAAGGATG CTCTTTCGAA CAGTGTTGCA TCTTCTGTCA GTAGATGTTA |
| 1201 | GTACTGCAGA GATGATGCCA GAAAATCTTA GGAAAAATTT AACTGAATTG CTTAGAGCAG |
| 1261 | CTTTAAAAAT TAGAATATGC CTAGAAAAGC AGCCTGACCC TTTTGCACCA AGACAAAAGA |
| 1321 | AAACACTGCA GGAGGTTCAG GAAGATTTTG TGTTTTCAAA GTATCGTCAT AGAGCCCTTC |
| 1381 | TTTTACCTGA GCTTTTGGAA GGAGTTCTTC AGATTCTGAT CTGTTGTCTT CAAAGTGCAG |
| 1441 | CTTCAAATCC CTTCTACTTC AGTCAAGCCA TGGATTTGGT TCAAGAATTC ATTCAGCATC |
| 1501 | ATGGATTTAA TTTATTTGAA ACAGCAGTTC TTCAAATGGA ATGGCTGGTT TTAAGAGATG |
| 1561 | GAGTTCCTCC CGAGGCCTCA GAGCATTTGA AAGCCCTAAT AAATAGTGTG ATGAAAATAA |
| 1621 | TGAGCACTGT CAAAAAAGTG AAATCAGAGC AACTTCATCA TTCGATGTGT ACAAGAAAAA |
| 1681 | GGCACAGACG ATGTGAATAT TCTCATTTTA TGCATCATCA CCGAGATCTC TCAGGTCTTC |
| 1741 | TGGTTTCGGC TTTTAAAAAC CAGGTTTCCA AAAACCCATT TGAAGAGACT GCAGATGGAG |
| 1801 | ATGTTTATTA TCCTGAGCGG TGCTGTTGCA TTGCAGTGTG TGCCCATCAG TGCTTGCGCT |
| 1861 | TACTACAGCA GGCTTCCTTG AGCAGCACTT GTGTCCAGAT CCTATCGGGT GTTCATAACA |
| 1921 | TTGGAATATG CTGTTGTATG GATCCCAAAT CTGTAATCAT TCCTTTGCTC CATGCTTTTA |
| 1981 | AATTGCCAGC ACTGAAAAAT TTTCAGCAGC ATATATTGAA TATCCTTAAC AAACTTATTT |
| 2041 | TGGATCAGTT AGGAGGAGCA GAGATATCAC CAAAAATTAA AAAAGCAGCT TGTAATATTT |
| 2101 | GTACTGTTGA CTCTGACCAA CTAGCCCAAT TAGAAGAGAC ACTGCAGGGA AACTTATGTG |
| 2161 | ATGCTGAACT CTCCTCAAGT TTATCCAGTC CTTCTTACAG ATTTCAAGGG ATCCTGCCCA |
| 2221 | GCAGTGGATC TGAAGATTTG TTGTGGAAAT GGGATGCTTT AAAGGCTTAT CAGAACTTTG |
| 2281 | TTTTTGAAGA AGACAGATTA CATAGTATAC AGATTGCAAA TCACATTTGC AATTTAATCC |
| 2341 | AGAAAGGCAA TATAGTTGTT CAGTGGAAAT TATATAATTA CATATTTAAT CCTGTGCTCC |
| 2401 | AAAGAGGAGT TGAATTAGCA CATCATTGTC AACACCTAAG CGTTACTTCA GCTCAAAGTC |
| 2461 | ATGTATGTAG CCATCATAAC CAGTGCTTGC CTCAGGACGT GCTTCAGATT TATGTAAAAA |
| 2521 | CTCTGCCTAT CCTGCTTAAA TCCAGGGTAA TAAGAGATTT GTTTTTGAGT TGTAATGGAG |
| 2581 | TAAGTCAAAT AATCGAATTA AATTGCTTAA ATGGTATTCG AAGTCATTCT CTAAAAGCAT |
| 2641 | TTGAAACTCT GATAATCAGC CTAGGGGAGC AACAGAAAGA TGCCTCAGTT CCAGATATTG |
| 2701 | ATGGGATAGA CATTGAACAG AAGGAGTTGT CCTCTGTACA TGTGGGTACT TCTTTTCATC |
| 2761 | ATCAGCAAGC TTATTCAGAT TCTCCTCAGA GTCTCAGCAA ATTTTATGCT GGCCTCAAAG |
| 2821 | AAGCTTATCC AAAGAGACGG AAGACTGTTA ACCAAGATGT TCATATCAAC ACAATAAACC |
| 2881 | TATTCCTCTG TGTGGCTTTT TTATGCGTAA GTAAAGAAGC AGAGTCTGAC AGGGAGTCGG |
| 2941 | CCAATGACTC AGAAGATACT TCTGGCTATG ACAGCACAGC CAGCGAGCCT TTAAGTCATA |
| 3001 | TGCTGCCATG TATATCTCTC GAGAGCCTTG TCTTGCCTTC TCCTGAACAT ATGCACCAAG |
| 3061 | CAGCAGACAT TTGGTCTATG TGTCGTTGGA TCTACATGTT GAGTTCAGTG TTCCAGAAAC |
| 3121 | AGTTTTATAG GCTTGGTGGT TTCCGAGTAT GCCATAAGTT AATATTTATG ATAATACAGA |
| 3181 | AACTGTTCAG AAGTCACAAA GAGGAGCAAG GAAAAAAGGA GGGAGATACA AGTGTAAATG |
| 3241 | AAAACCAGGA TTTAAACAGA ATTTCTCAAC CTAAGAGAAC TATGAAGGAA GATTTATTAT |
| 3301 | CTTTGGCTAT AAAAAGTGAC CCCATACCAT CAGAACTAGG TAGTCTAAAA AAGAGTGCTG |
| 3361 | ACAGTTTAGG TAAATTAGAG TTACAGCATA TTTCTTCCAT AAATGTGGAA GAAGTTTCAG |
| 3421 | CTACTGAAGC CGCTCCCGAG GAAGCAAAGC TATTTACAAG TCAAGAAAGT GAGACCTCAC |
| 3481 | TTCAAAGTAT ACGACTTTTG GAAGCCCTTC TGGCCATTTG TCTTCATGGT GCCAGAACTA |
| 3541 | GTCAACAGAA GATGGAATTG GAGTTACCTA ATCAGAACTT GTCTGTGGAA AGTATATTAT |
| 3601 | TTGAAATGAG GGACCATCTT TCCCAGTCAA AGGTGATTGA AACACAACTA GCAAAGCCTT |
| 3661 | TATTTGATGC CCTGCTTCGA GTTGCCCTCG GGAATTATTC AGCAGATTTT GAACATAATG |
| 3721 | ATGCTATGAC TGAGAAGAGT CATCAATCTG CAGAAGAATT GTCATCCCAG CCTGGTGATT |
| 3781 | TTTCAGAAGA AGCTGAGGAT TCTCAGTGTT GTAGTTTTAA ACTTTTAGTT GAAGAAGAAG |
| 3841 | GTTACGAAGC AGATAGTGAA AGCAATCCTG AAGATGGCGA AACCCAGGAT GATGGGGTAG |
| 3901 | ACTTAAAGTC TGAAACAGAA GGTTTCAGTG CATCAAGCAG TCCAAATGAC TTACTCGAAA |
| 3961 | ACCTCACTCA AGGGGAAATA ATTTATCCTG AGATTTGTAT GCTGGAATTA AATTTGCTTT |
| 4021 | CTGCTAGTAA AGCCAAACTT GATGTGCTTG CCCATGTATT TGAGAGTTTT TTGAAAATTA |
| 4081 | TTAGGCAGAA AGAAAAGAAT GTTTTTCTGC TCATGCAACA GGGAACTGTG AAAAATCTTT |
| 4141 | TAGGAGGGTT CTTGAGTATT TTAACACAGG ATGATTCTGA TTTTCAAGCA TGCCAGAGAG |
| 4201 | TATTGGTGGA TCTTTTGGTA TCTTTGATGA GTTCAAGAAC ATGTTCAGAA GAGCTAACCC |
| 4261 | TTCTTTTGAG AATATTTCTG GAGAAATCTC CTTGTACAAA AATTCTTCTT CTGGGTATTC |
| 4321 | TGAAAATTAT TGAAAGTGAT ACTACTATGA GCCCTTCACA GTATCTAACC TTCCCTTTAC |
| 4381 | TGCACGCTCC AAATTTAAGC AACGGTGTTT CATCACAAAA GTATCCTGGG ATTTTAAACA |
| 4441 | GTAAGGCCAT GGGTTTATTG AGAAGAGCAC GAGTTTCACG GAGCAAGAAA GAGGCTGATA |
| 4501 | GAGAGAGTTT TCCCCATCGG CTGCTTTCAT CTTGGCACAT AGCCCCAGTC CACCTGCCGT |
| 4561 | TGCTGGGGCA AAACTGCTGG CCACACCTAT CAGAAGGTTT CAGTGTTTCC CTGTGGTTTA |
| 4621 | ATGTGGAGTG TATCCATGAA GCTGAGAGTA CTACAGAAAA AGGAAAGAAG ATAAAGAAAA |
| 4681 | GAAACAAATC ATTAATTTTA CCAGATAGCA GTTTTGATGG TACAGAGAGC GACAGACCAG |
| 4741 | AAGGTGCAGA GTACATAAAT CCTGGTGAAA GACTCATAGA AGAAGGATGT ATTCATATAA |
| 4801 | TTTCACTGGG ATCCAAAGCG TTGATGATCC AAGTGTGGGC TGATCCCCAC AATGCCACTC |
| 4861 | TTATCTTTCG TGTGTGCATG GATTCAAATG ATGACATGAA AGCTGTTTTA CTAGCACAGG |
| 4921 | TTGAATCACA GGAGAATATT TTCCTCCCAA GCAAATGGCA ACATTTAGTA CTCACCTACT |
| 4981 | TACAGCAGCC CCAAGGGAAA AGGAGGATTC ATGGGAAAAT CTCCATATGG GTCTCTGGAC |
| 5041 | AGAGGAAGCC TGATGTTACT TTGGATTTTA TGCTTCCAAG AAAAACAAGT TTGTCATCTG |
| 5101 | ATAGCAATAA AACATTTTGC ATGATTGGCC ATTGTTTATC ATCCCAAGAA GAGTTTTTGC |
| 5161 | AGTTGGCTGG AAAATGGGAC CTGGGAAATT TGCTTCTCTT CAACGGAGCT AAGGTTGGTT |
| 5221 | CACAAGAGGC CTTTTATCTG TATGCTTGTG GACCCAACCA TACATCTGTA ATGCCATGTA |
| 5281 | AGTATGGCAA GCCAGTCAAT GACTACTCCA AATATATTAA TAAAGAAATT TTGCGATGTG |
| 5341 | AACAAATCAG AGAACTTTTT ATGACCAAGA AAGATGTGGA TATTGGTCTC TTAATTGAAA |
| 5401 | GTCTTTCAGT TGTTTATACA ACTTACTGTC CTGCTCAGTA TACCATCTAT GAACCAGTGA |
| 5461 | TTAGACTTAA AGGTCAAATG AAAACCCAAC TCTCTCAAAG ACCCTTCAGC TCAAAAGAAG |
| 5521 | TTCAGAGCAT CTTATTAGAA CCTCATCATC TAAAGAATCT CCAACCTACT GAATATAAAA |
| 5581 | CTATTCAAGG CATTCTGCAC GAAATTGGTG GAACTGGCAT ATTTGTTTTT CTCTTTGCCA |
| 5641 | GGGTTGTTGA ACTCAGTAGC TGTGAAGAAA CTCAAGCATT AGCACTGCGA GTTATACTCT |
| 5701 | CATTAATTAA ATACAACCAA CAAAGAGTAC ATGAATTAGA AAATTGTAAT GGACTTTCTA |
| 5761 | TGATTCATCA GGTGTTGATC AAACAAAAAT GCATTGTTGG GTTTTACATT TTGAAGACCC |
| 5821 | TTCTTGAAGG ATGCTGTGGT GAAGATATTA TTTATATGAA TGAGAATGGA GAGTTTAAGT |
| 5881 | TGGATGTAGA CTCTAATGCT ATAATCCAAG ATGTTAAGCT GTTAGAGGAA CTATTGCTTG |
| 5941 | ACTGGAAGAT ATGGAGTAAA GCAGAGCAAG GTGTTTGGGA AACTTTGCTA GCAGCTCTAG |
| 6001 | AAGTCCTCAT CAGAGCAGAT CACCACCAGC AGATGTTTAA TATTAAGCAG TTATTGAAAG |
| 6061 | CTCAAGTGGT TCATCACTTT CTACTGACTT GTCAGGTTTT GCAGGAATAC AAAGAGGGGC |
| 6121 | AACTCACACC CATGCCCCGA GAGGTTTGTA GATCATTTGT GAAAATTATA GCAGAAGTCC |
| 6181 | TTGGATCTCC TCCAGATTTG GAATTATTGA CAATTATCTT CAATTTCCTT TTAGCAGTTC |
| 6241 | ACCCTCCTAC TAATACTTAC GTTTGTCACA ATCCCACGAA CTTCTACTTT TCTTTGCACA |
| 6301 | TAGATGGCAA GATCTTTCAG GAGAAAGTGC GGTCAATCAT GTACCTGAGG CATTCCAGCA |
| 6361 | GTGGAGGAAG GTCCCTTATG AGCCCTGGAT TTATGGTAAT AAGCCCATCT GGTTTTACTG |
| 6421 | CTTCACCATA TGAAGGAGAG AATTCCTCTA ATATTATTCC ACAACAGATG GCCGCCCATA |
| 6481 | TGCTGCGTTC TAGAAGCCTA CCAGCATTCC CTACTTCTTC ACTACTAACG CAATCACAAA |
| 6541 | AACTGACTGG AAGTTTGGGT TGTAGTATCG ACAGGTTACA AAATATTGCA GATACTTATG |
| 6601 | TTGCCACCCA ATCAAAGAAA CAAAATTCTT TGGGGAGTTC CGACACACTG AAAAAAGGCA |
| 6661 | AAGAGGACGC ATTCATCAGT AGCTGTGAGT CTGCAAAAAC TGTTTGTGAA ATGGAAGCTG |
| 6721 | TCCTCTCAGC CCAGGTCTCT GTCAGTGATG TCCCAAAGGG AGTGCTGGGA TTTCCAGTGG |
| 6781 | TCAAAGCAGA TCATAAACAG TTGGGAGCAG AACCCAGGTC AGAAGATGAC AGTCCTGGGG |
| 6841 | ATGAGTCCTG CCCACGCCGA CCTGATTACC TAAAGGGATT GGCCTCCTTC CAGCGAAGCC |
| 6901 | ACAGCACTAT TGCAAGCCTT GGGCTAGCTT TTCCTTCACA GAACGGATCT GCAGCTGTTG |
| 6961 | GCCGTTGGCC AAGTCTTGTT GATAGAAACA CTGATGATTG GGAAAACTTT GCCTATTCTC |
| 7021 | TTGGTTATGA GCCAAATTAC AACCGAACTG CAAGTGCTCA CAGTGTAACT GAAGACTGTT |
| 7081 | TGGTACCTAT ATGCTGTGGA TTATATGAAC TCCTAAGTGG GGTTCTTCTT ATCCTGCCTG |
| 7141 | ATGTTTTGCT TGAAGATGTG ATGGACAAGC TTATTCAAGC AGATACACTT TTGGTCCTCG |
| 7201 | TTAACCACCC ATCACCAGCT ATACAACAAG GTGTTATTAA ACTATTAGAT GCATATTTTG |
| 7261 | CTAGAGCATC TAAGGAACAA AAAGATAAAT TTCTGAAGAA TCGTGGATTT TCCTTGCTAG |
| 7321 | CCAACCAGTT GTATCTTCAT CGAGGAACTC AAGAATTGTT AGAATGCTTC ATCGAAATGT |
| 7381 | TCTTTGGTCG ACATATTGGC CTTGATGAAG AATTTGATCT GGAAGATGTG AGAAACATGG |
| 7441 | GATTGTTTCA GAAGTGGTCT GTCATTCCTA TTCTGGGACT AATAGAGACC TCTCTATATG |
| 7501 | ACAACATACT CTTGCATAAT GCTCTTTTAC TTCTTCTCCA AATTTTAAAT TCTTGTTCTA |
| 7561 | AGGTAGCAGA TATGTTGCTG GATAATGGTC TACTCTATGT GTTATGTAAT ACAGTAGCAG |
| 7621 | CCCTGAATGG ATTAGAAAAG AACATTCCCA TGAGTGAATA TAAATTGCTT GCTTGTGATA |
| 7681 | TACAGCAACT TTTCATAGCA GTTACAATTC ATGCTTGCAG TTCCTCAGGC TCACAATATT |
| 7741 | TTAGGGTTAT TGAAGACCTT ATTGTAATGC TTGGATATCT TCAAAATAGC AAAAACAAGA |
| 7801 | GGACACAAAA TATGGCTGTT GCACTACAGC TTAGAGTTCT CCAGGCTGCT ATGGAATTTA |
| 7861 | TAAGGACCAC CGCAAATCAT GACTCTGAAA ACCTCACAGA TTCACTCCAG TCACCTTCTG |
| 7921 | CTCCCCATCA TGCAGTAGTT CAAAAGCGGA AAAGCATTGC TGGTCCTCGA AAATTTCCCC |
| 7981 | TTGCTCAAAC TGAATCGCTT CTGATGAAAA TGCGTTCAGT GGCAAATGAT GAGCTTCATG |
| 8041 | TGATGATGCA ACGGAGAATG AGCCAAGAGA ACCCTAGCCA AGCAACTGAA ACGGAACTTG |
| 8101 | CGCAGAGACT ACAGAGGCTC ACTGTTTTAG CAGTCAACAG GATTATTTAT CAAGAATTTA |
| 8161 | ATTCAGACAT TATTGACATT TTGAGAACTC CAGAAAATGT AACTCAAAGC AAGACCTCAG |
| 8221 | TTTTCCAGAC CGAAATTTCT GAGGAAAATA TTCATCATGA ACAGTCTTCT GTTTTCAATC |
| 8281 | CATTTCAGAA AGAAATTTTT ACATATCTGG TAGAAGGATT CAAAGTATCT ATTGGTTCAA |
| 8341 | GTAAAGCCAG TGGTTCCAAG CAGCAATGGA CTAAAATTCT GTGGTCTTGT AAGGAGACCT |
| 8401 | TCCGAATGCA GCTTGGGAGA CTACTAGTGC ATATTTTGTC GCCAGCCCAC GCTGCACAAG |
| 8461 | AGAGAAAGCA AATTTTTGAA ATAGTTCATG AACCAAATCA TCAGGAAATA CTACGAGACT |
| 8521 | GTCTCAGCCC ATCCCTACAA CATGGAGCCA AGTTAGTTTT GTATTTGTCA GAGTTGATAC |
| 8581 | ATAATCACCA AGGTGAATTG ACTGAAGAAG AGCTAGGCAC AGCAGAACTG CTTATGAATG |
| 8641 | CTTTGAAGTT ATGTGGTCAC AAGTGCATCC CTCCCAGTGC ATCAACAAAA GCAGACCTTA |
| 8701 | TTAAAATGAT CAAAGAGGAA CAAAAGAAAT ATGAAACTGA AGAAGGAGTG AATAAAGCTG |
| 8761 | CTTGGCAGAA AACAGTTAAC AATAATCAAC AAAGTCTCTT TCAGCGTCTG GATTCAAAAT |
| 8821 | CAAAGGATAT ATCTAAAATA GCTGCAGATA TCACCCAGGC AGTGTCTCTC TCCCAAGGAA |
| 8881 | ATGAGAGAAA AAAGGTGATC CAGCATATTA GAGGAATGTA TAAAGTAGAT TTGAGTGCCA |
| 8941 | GCAGACATTG GCAGGAACTT ATTCAGCAGC TGACACATGA TAGAGCAGTA TGGTATGACC |
| 9001 | CCATCTACTA TCCAACCTCA TGGCAGTTGG ATCCAACAGA AGGGCCAAAT CGAGAGAGGA |
| 9061 | GACGTTTACA GAGATGTTAT TTAACTATTC CAAATAAGTA TCTCCTTAGG GATAGACAGA |
| 9121 | AATCAGAAGA TGTTGTCAAA CCACCACTCT CTTACCTGTT TGAAGACAAA ACTCATTCTT |
| 9181 | CTTTCTCTTC TACTGTCAAA GACAAAGCTG CAAGTGAATC TATAAGAGTG AATCGAAGAT |
| 9241 | GCATCAGTGT TGCACCATCT AGAGAGACAG CTGGTGAATT GTTACTAGGT AAATGTGGAA |
| 9301 | TGTATTTTGT GGAAGATAAT GCTTCTGATA CAGTTGAAAG TTCGAGCCTT CAGGGAGAGT |
| 9361 | TGGAACCAGC ATCATTTTCC TGGACATATG AAGAAATTAA AGAAGTTCAC AAGCGTTGGT |
| 9421 | GGCAATTGAG AGATAATGCT GTAGAAATCT TTCTAACAAA TGGCAGAACA CTCCTGTTGG |
| 9481 | CATTTGATAA CACCAAGGTT CGTGATGATG TATACCACAA TATACTCACA AATAACCTCC |
| 9541 | CTAATCTTCT GGAATATGGT AACATCACCG CTCTGACAAA TTTATGGTAT ACTGGGCAAA |
| 9601 | TTACTAATTT TGAATATTTG ACTCACTTAA ACAAACATGC TGGCCGATCC TTCAATGATC |
| 9661 | TCATGCAGTA TCCTGTGTTC CCATTTATAC TTGCTGACTA CGTTAGTGAG ACACTTGACC |
| 9721 | TCAATGATCT GTTGATATAC AGAAATCTCT CTAAACCTAT AGCTGTTCAG TATAAAGAAA |
| 9781 | AAGAAGATCG TTATGTGGAC ACATACAAGT ACTTGGAGGA AGAGTACCGC AAAGGAGCCA |
| 9841 | GAGAAGATGA CCCCATGCCT CCCGTGCAGC CCTATCACTA TGGCTCCCAC TATTCCAATA |
| 9901 | GCGGCACTGT GCTTCACTTC CTGGTCAGGA TGCCTCCTTT CACTAAAATG TTTTTAGCCT |
| 9961 | ATCAAGATCA AAGTTTTGAC ATTCCAGACA GAACTTTTCA TTCTACAAAT ACAACTTGGC |
| 10021 | GACTCTCATC TTTTGAATCT ATGACTGATG TGAAAGAACT TATCCCAGAG TTTTTCTATC |
| 10081 | TTCCAGAGTT CCTAGTTAAC CGTGAAGGTT TTGATTTTGG TGTGCGTCAG AATGGTGAAC |
| 10141 | GGGTTAATCA CGTCAACCTT CCCCCTTGGG CGCGTAATGA TCCTCGTCTT TTTATCCTCA |
| 10201 | TCCATCGGCA GGCTCTAGAG TCTGACTACG TGTCGCAGAA CATCTGTCAG TGGATTGACT |
| 10261 | TGGTGTTTGG GTATAAGCAA AAGGGGAAGG CTTCTGTTCA AGCGATCAAT GTTTTTCATC |
| 10321 | CTGCTACATA TTTTGGAATG GATGTCTCTG CAGTTGAAGA TCCAGTTCAG AGACGAGCGC |
| 10381 | TAGAAACCAT GATAAAAACC TACGGGCAGA CTCCCCGTCA GCTGTTCCAC ATGGCCCATG |
| 10441 | TGAGCAGACC TGGAGCCAAG CTCAATATTG AAGGAGAGCT TCCAGCTGCT GTGGGGTTGC |
| 10501 | TAGTGCAGTT TGCTTTCAGG GAGACCCGAG AACAGGTCAA AGAAATCACC TATCCGAGTC |
| 10561 | CTTTGTCATG GATAAAAGGC TTGAAATGGG GGGAATACGT GGGTTCCCCC AGTGCTCCAG |
| 10621 | TACCTGTGGT CTGCTTCAGC CAGCCCCACG GAGAAAGATT TGGCTCTCTC CAGGCTCTGC |
| 10681 | CCACCAGAGC AATCTGTGGT TTGTCACGGA ATTTCTGTCT TCTGATGACA TATAGCAAGG |
| 10741 | AACAAGGTGT GAGAAGCATG AACAGTACGG ACATTCAGTG GTCAGCCATC CTGAGCTGGG |
| 10801 | GATATGCTGA TAATATTTTA AGGTTGAAGA GTAAACAAAG TGAGCCTCCA GTAAACTTTA |
| 10861 | TTCAAAGTTC ACAACAGTAC CAGGTGACTA GTTGTGCTTG GGTGCCTGAC AGTTGCCAGC |
| 10921 | TGTTTACTGG AAGCAAATGC GGTGTCATCA CAGCCTACAC AAACAGATTT ACAAGCAGCA |
| 10981 | CGCCATCAGA AATAGAAATG GAGACTCAAA TACATCTCTA TGGTCACACA GAAGAGATAA |
| 11041 | CCAGCTTATT TGTTTGCAAA CCATACAGTA TACTGATAAG TGTGAGCAGA GACGGAACCT |
| 11101 | GCATCATATG GGATTTAAAC AGGTTATGCT ATGTACAAAG TCTGGCGGGA CACAAAAGCC |
| 11161 | CTGTCACAGC TGTCTCTGCC AGTGAAACCT CAGGTGATAT TGCTACTGTG TGTGATTCAG |
| 11221 | CTGGCGGAGG CAGTGACCTC AGACTCTGGA CGGTGAACGG GGATCTCGTT GGACATGTCC |
| 11281 | ACTGCAGGGA GATCATCTGT TCCGTGGCTT TCTCCAACCA GCCTGAGGGA GTATCTATCA |
| 11341 | ATGTAATCGC TGGGGGATTA GAAAATGGAA TTGTAAGGTT ATGGAGCACA TGGGACTTAA |
| 11401 | AGCCTGTGAG AGAAATTACA TTTCCCAAAT CAAATAAGCC CATCATCAGC CTTACATTTT |
| 11461 | CTTGTGATGG CCACCATTTG TACACAGCAA ACAGTGATGG GACCGTGATT GCCTGGTGTC |
| 11521 | GGAAGGACCA GCAGCGCTTG AAACAGCCAA TGTTCTATTC CTTCCTTAGC AGCTATGCAG |
| 11581 | CCGGGTGAAT GCGAATGAAC TTCACGTTCT CCAAAGCACT TTAACTCCAA ACTAGATTTG |
| 11641 | TTGACTTCAC CAGTTTTAGG AGGTTGAACC TAAAGAAATG GATGACTGGA CAAACCATCC |
| 11701 | AAATAATGAT AAAGTCTATT CATCTGCACA AAATTCTGAA GAGTCACATG ATCCTAAGAG |
| 11761 | GAAAGTTCTG TTCTATTTTA GTGATAATCT GGAAGATTGT GTCAATATGC ACTAGCCAAC |
| 11821 | AAGTTTTAAG CCTCGCATGG TACATTAAAA TGATATTCTT AAAATTTTTT CCCACCAAGG |
| 11881 | TATTCCAAAG AAAATATTAA GGTCTCCCCT TTTTCTATGA TTCCAAAAGG ACCAGTAGAA |
| 11941 | TTTAAATTGG TTGGTTGATT GTTTATATAA AACACACTAA AATTATATTT TAAAAGTTTA |
| 12001 | CTGCCATGAA ATACTCCTCC CACCACACAC ACATGCTCCA AAAGAGGAAA GAAAAAAAGA |
| 12061 | TAATTTTTAG GACTTGATAA TTGCTTTCTT TGAGAAGCAA ATTATTCAGT AGGTGCCTCT |
| 12121 | GTACCAAATA TTTTATGGAA TATCTAAATA CTAAAATAAA CTATGAATGA ATCTCAAAAT |
| 12181 | TAGGCAGTTT TTGCCAGTTG CTTTCTTAGC TCAAAGGAGA ACCAGAATTT TTTTGACAGC |
| 12241 | CACAAACAAG AATACAGGTA TCTTGGATTT CAGACACATT CTGTTTCTTC ATAAAAATTT |
| 12301 | TACTTAAAAT CTGTAACGCT AGATATTGAC TATCCTTAGT TGAGTCACTG AGGTTTAAAC |
| 12361 | ACAATGGTAA GTCTTAAAGT CTGCTATTTA CAGAGCATTG AATCTGTACC AATTTGCAAT |
| 12421 | AGAAAGCCTT CAGTATGCAA GAAGTTTGCA TGGGTATTAA GAACACAGCC TAAATAAGGC |
| 12481 | ATTTGATCTA ATCTGCAGGA AGAATTTTCT TCCCCAAAAC AGAATTATAA AAGCTTACTT |
| 12541 | TAAACAGGAG GCAGAATAAT TCTTTTAGGA AACCATTTCA TTCTGTTTCT ACTAACCTAT |
| 12601 | ACCATCTGAG AATTCTAGGA AGAATAATAA AATCTCGTGT ATTCCACAGC AAACTTACAT |
| 12661 | ACCATAAAGA CAGAATTCCT AAACATCTTG GAGCCGTCTG TCTCTCCCAT ATGATGGCTG |
| 12721 | TCTGTATATT TTTACTTGGG GTGCTGCTTT ATTGGCTTTG AAAACACTGT CAGATAAGCT |
| 12781 | CAGTAATATG TTACCATGGG ATAAAAATAT GTATCCCTGC CTAAGAATAA CTTGTGCATT |
| 12841 | TGTTATGGAA ATTTAATTCA TATGGTGTTT ACAGTACTAC TTTTGTAACT TCCAGACTTT |
| 12901 | CTAAAACATT CTGCTTAAAA ACCATATAAA ATATAATTCC AAAGTCTCTG CTGTCAAGAT |
| 12961 | AGATTCGAGA GAAAGCACGT GGCCATGTAT GCTTTAACCT TAAACTGCAT ACACATGTAG |
| 13021 | TGATACCTAG GCTGCATTTA GATCACCGTG TGCTCAGGCC AGGTGTGAAT CCTGAGGTCC |
| 13081 | ATGGAGGTGC AGAGATGAGA TTACTCCTAT TCACGTTGAA GTGATTTGCT TTGTTAACAA |
| 13141 | AAAATTGCAG CTATTGTCTA GCTTTCATTT TTTTACTGAG AACTTTAAAT TAGTCCCCTA |
| 13201 | TTAGAATAGG GTTGCTACTC ATCTTTTTTT AAAAACCGAA TTTCATCATT TATCTAAAGA |
| 13261 | GAAAATATGC AGAATAACTG GTCTTGTTAA GAGTGCAATA TTATATTTTT ATGTAAAAAT |
| 13321 | AAAAATTAAT TTGGGGGGAT TATTTATTCA GCATGAAACC TAATATGTAT ATGTTTGAAA |
| 13381 | TACTTCATAA TGTGCATGTT GTAGCAAACA TTTCTGTAAA TTATCACAAG CTCTGTTACC |
| 13441 | TTTATATACA CTGCCTCTTC AATTTGGAAA TAAATTTCAT AAAAATAGAT GTAAACAAAA |
| 13501 | AAA. |

Nucleotide sequences that have at least 80%, 85%, 90%, 95%, 99% or 100%
amino acid sequence identity to SEQ ID NO: 1 are also disclosed.

2. The LYST Protein

The LYST polypeptide is a 3,801 amino acid cytoplasmic protein (also known as lysosomal trafficking regulator, CHS protein, CHS1 or LYST protein) with a molecular weight of approximately 429 kDa (Barbosa, et al., Nature, 382(6588): 262-265 (1996)).

The LYST protein exists as one of three isoforms and is predicted to adopt a helical structure (Barbosa, et al., Nature, 382(6588): 262-265 (1996)).

The LYST protein is highly conserved across mammalian species and is expressed at low levels in all cell types, but is abundantly expressed in adult and fetal thymus, peripheral blood leukocytes, bone marrow and several regions of adult brain (see review by Dotta, et al., Orphanet Journal of Rare Diseases, 8:168 (2013), and references therein).

Amino acid sequences of the human LYST protein are known in the art. See, for example, GenBank Accession No. U67615:

| (SEQ ID NO: 2) | |
|---|---|
| MSTDSNSLAR EFLTDVNRLC NAVVQRVEAR EEEEEETHMA TLGQYLVHGR GFLLLTKLNS | 60 |
| IIDQALTCRE ELLTLLLSLL PLVWKIPVQE EKATDFNLPL SADIILTKEK NSSSQRSTQE | 120 |
| KLHLEGSALS SQVSAKVNVF RKSRRQRKIT HRYSVRDARK TQLSTSDSEA NSDEKGIAMN | 180 |
| KHRRPHLLHH FLTSFPKQDH PKAKLDRLAT KEQTPPDAMA LENSREIIPR QGSNTDILSE | 240 |
| PAALSVISNM NNSPFDLCHV LLSLLEKVCK FDVTLNHNSP LAASVVPTLT EFLAGFGDCC | 300 |
| SLSDNLESRV VSAGWTEEPV ALIQRMLFRT VLHLLSVDVS TAEMMPENLR KNLTELLRAA | 360 |
| LKIRICLEKQ PDPFAPRQKK TLQEVQEDFV FSKYRHRALL LPELLEGVLQ ILICCLQSAA | 420 |
| SNPFYFSQAM DLVQEFIQHH GFNLFETAVL QMEWLVLRDG VPPEASEHLK ALINSVMKIM | 480 |
| STVKKVKSEQ LHHSMCTRKR HRRCEYSHFM HHHRDLSGLL VSAFKNQVSK NPFEETADGD | 540 |
| VYYPERCCCI AVCAHQCLRL LQQASLSSTC VQILSGVHNI GICCCMDPKS VIIPLLHAFK | 600 |
| LPALKNFQQH ILNILNKLIL DQLGGAEISP KIKKAACNIC TVDSDQLAQL EETLQGNLCD | 660 |
| AELSSSLSSP SYRFQGILPS SGSEDLLWKW DALKAYQNFV FEEDRLHSIQ IANHICNLIQ | 720 |
| KGNIVVQWKL YNYIFNPVLQ RGVELAHHCQ HLSVTSAQSH VCSHHNQCLP QDVLQIYVKT | 780 |
| LPILLKSRVI RDLFLSCNGV SQIIELNCLN GIRSHSLKAF ETLIISLGEQ QKDASVPDID | 840 |
| GIDIEQKELS SVHVGTSFHH QQAYSDSPQS LSKFYAGLKE AYPKRRKTVN QDVHINTINL | 900 |
| FLCVAFLCVS KEAESDRESA NDSEDTSGYD STASEPLSHM LPCISLESLV LPSPEHMHQA | 960 |
| ADIWSMCRWI YMLSSVFQKQ FYRLGGFRVC HKLIFMIIQK LFRSHKEEQG KKEGDTSVNE | 1020 |
| NQDLNRISQP KRTMKEDLLS LAIKSDPIPS ELGSLKKSAD SLGKLELQHI SSINVEEVSA | 1080 |
| TEAAPEEAKL FTSQESETSL QSIRLLEALL AICLHGARTS QQKMELELPN QNLSVESILF | 1140 |
| EMRDHLSQSK VIETQLAKPL FDALLRVALG NYSADFEHND AMTEKSHQSA EELSSQPGDF | 1200 |
| SEEAEDSQCC SFKLLVEEEG YEADSESNPE DGETQDDGVD LKSETEGFSA SSSPNDLLEN | 1260 |
| LTQGEIIYPE ICMLELNLLS ASKAKLDVLA HVFESFLKII RQKEKNVFLL MQQGTVKNLL | 1320 |
| GGFLSILTQD DSDFQACQRV LVDLLVSLMS SRTCSEELTL LLRIFLEKSP CTKILLLGIL | 1380 |
| KIIESDTTMS PSQYLTFPLL HAPNLSNGVS SQKYPGILNS KAMGLLRRAR VSRSKKEADR | 1440 |
| ESFPHRLLSS WHIAPVHLPL LGQNCWPHLS EGFSVSLWFN VECIHEAEST TEKGKKIKKR | 1500 |
| NKSLILPDSS FDGTESDRPE GAEYINPGER LIEEGCIHII SLGSKALMIQ VWADPHNATL | 1560 |
| IFRVCMDSND DMKAVLLAQV ESQENIFLPS KWQHLVLTYL QQPQGKRRIH GKISIWVSGQ | 1620 |
| RKPDVTLDFM LPRKTSLSSD SNKTFCMIGH CLSSQEEFLQ LAGKWDLGNL LLFNGAKVGS | 1680 |
| QEAFYLYACG PNHTSVMPCK YGKPVNDYSK YINKEILRCE QIRELFMTKK DVDIGLLIES | 1740 |
| LSVVYTTYCP AQYTIYEPVI RLKGQMKTQL SQRPFSSKEV QSILLEPHHL KNLQPTEYKT | 1800 |
| IQGILHEIGG TGIFVFLFAR VVELSSCEET QALALRVILS LIKYNQQRVH ELENCNGLSM | 1860 |
| IHQVLIKQKC IVGFYILKTL LEGCCGEDII YMNENGEFKL DVDSNAIIQD VKLLEELLLD | 1920 |
| WKIWSKAEQG VWETLLAALE VLIRADHHQQ MFNIKQLLKA QVVHHFLLTC QVLQEYKEGQ | 1980 |
| LTPMPREVCR SFVKIIAEVL GSPPDLELLT IIFNFLLAVH PPTNTYVCHN PTNFYFSLHI | 2040 |
| DGKIFQEKVR SIMYLRHSSS GGRSLMSPGF MVISPSGFTA SPYEGENSSN IIPQQMAAHM | 2100 |
| LRSRSLPAFP TSSLLTQSQK LTGSLGCSID RLQNIADTYV ATQSKKQNSL GSSDTLKKGK | 2160 |
| EDAFISSCES AKTVCEMEAV LSAQVSVSDV PKGVLGFPVV KADHKQLGAE PRSEDDSPGD | 2220 |
| ESCPRRPDYL KGLASFQRSH STIASLGLAF PSQNGSAAVG RWPSLVDRNT DDWENFAYSL | 2280 |
| GYEPNYNRTA SAHSVTEDCL VPICCGLYEL LSGVLLILPD VLLEDVMDKL IQADTLLVLV | 2340 |
| NHPSPAIQQG VIKLLDAYFA RASKEQKDKF LKNRGFSLLA NQLYLHRGTQ ELLECFIEMF | 2400 |
| FGRHIGLDEE FDLEDVRNMG LFQKWSVIPI LGLIETSLYD NILLHNALLL LLQILNSCSK | 2460 |
| VADMLLDNGL LYVLCNTVAA LNGLEKNIPM SEYKLLACDI QQLFIAVTIH ACSSSGSQYF | 2520 |
| RVIEDLIVML GYLQNSKNKR TQNMAVALQL RVLQAAMEFI RTTANHDSEN LTDSLQSPSA | 2580 |
| PHHAVVQKRK SIAGPRKFPL AQTESLLMKM RSVANDELHV MMQRRMSQEN PSQATETELA | 2640 |
| QRLQRLTVLA VNRIIYQEFN SDIIDILRTP ENVTQSKTSV FQTEISEENI HHEQSSVFNP | 2700 |
| FQKEIFTYLV EGFKVSIGSS KASGSKQQWT KILWSCKETF RMQLGRLLVH ILSPAHAAQE | 2760 |
| RKQIFEIVHE PNHQEILRDC LSPSLQHGAK LVLYLSELIH NHQGELTEEE LGTAELLMNA | 2820 |
| LKLCGHKCIP PSASTKADLI KMIKEEQKKY ETEEGVNKAA WQKTVNNNQQ SLFQRLDSKS | 2880 |
| KDISKIAADI TQAVSLSQGN ERKKVIQHIR GMYKVDLSAS RHWQELIQQL THDRAVWYDP | 2940 |
| IYYPTSWQLD PTEGPNRERR RLQRCYLTIP NKYLLRDRQK SEDVVKPPLS YLFEDKTHSS | 3000 |
| FSSTVKDKAA SESIRVNRRC ISVAPSRETA GELLLGKCGM YFVEDNASDT VESSSLQGEL | 3060 |
| EPASFSWTYE EIKEVHKRWW QLRDNAVEIF LTNGRTLLLA FDNTKVRDDV YHNILTNNLP | 3120 |
| NLLEYGNITA LTNLWYTGQI TNFEYLTHLN KHAGRSFNDL MQYPVFPFIL ADYVSETLDL | 3180 |
| NDLLIYRNLS KPIAVQYKEK EDRYVDTYKY LEEEYRKGAR EDDPMPPVQP YHYGSHYSNS | 3240 |
| GTVLHFLVRM PPFTKMFLAY QDQSFDIPDR TFHSTNTTWR LSSFESMTDV KELIPEFFYL | 3300 |
| PEFLVNREGF DFGVRQNGER VNHVNLPPWA RNDPRLFILI HRQALESDYV SQNICQWIDL | 3360 |
| VFGYKQKGKA SVQAINVFHP ATYFGMDVSA VEDPVQRRAL ETMIKTYGQT PRQLFHMAHV | 3420 |
| SRPGAKLNIE GELPAAVGLL VQFAFRETRE QVKEITYPSP LSWIKGLKWG EYVGSPSAPV | 3480 |
| PVVCFSQPHG ERFGSLQALP TRAICGLSRN FCLLMTYSKE QGVRSMNSTD IQWSAILSWG | 3540 |
| YADNILRLKS KQSEPPVNFI QSSQQYQVTS CAWVPDSCQL FTGSKCGVIT AYTNRFTSST | 3600 |
| PSEIEMETQI HLYGHTEEIT SLFVCKPYSI LISVSRDGTC IIWDLNRLCY VQSLAGHKSP | 3660 |
| VTAVSASETS GDIATVCDSA GGGSDLRLWT VNGDLVGHVH CREIICSVAF SNQPEGVSIN | 3720 |
| VIAGGLENGI VRLWSTWDLK PVREITFPKS NKPIISLTFS CDGHHLYTAN SDGTVIAWCR | 3780 |
| KDQQRLKQPM FYSFLSSYAA G | 3801 |

LYST polypeptides that have at least 80%, 85%, 90%, 95%, 99% or 100% amino acid sequence identity to SEQ ID NO: 2 are disclosed.

Although the LYST protein has been associated with many diverse cellular activities, particularly in intracellular protein trafficking in endosomes and lysosomes, the biological function of the LYST protein has remained largely unknown.

Abnormal expression and function of the LYST gene product has been implicated in numerous pathological disorders, including autoimmune diseases, hyperproliferative disorders and platelet dysfunction. Specifically, mutations in the LYST gene are associated with the human disease Chediak-Higashi syndrome (CHS) (Barrat, et al., Am. J. Hum. Genet, 59:625-632 (1996)). In the CHS patient, the LYST gene contains a frame-shift mutation at nucleotides 117-118 of the coding domain.

CHS is a rare lysosomal storage disorder characterized by hypopigmentation, severe immunologic deficiency, a bleeding tendency, neurologic abnormalities, abnormal intracellular transport to and from the lysosome and giant inclusion bodies in a variety of cell types. At least 30 mutations in the LYST gene have been identified in people with Chediak-Higashi syndrome. These mutations impair the normal function of the lysosomal trafficking regulator protein, which disrupts the size, structure, and function of Iysosomes and related structures within cells. People with LYST mutations have abnormally large lysosomes and related structures in cells throughout the body. These enlarged structures interfere with normal cell functions. Enlarged lysosomes in immune system cells, such as macrophages, prevent these cells from responding appropriately to bacteria and other foreign invaders; large perinuclear lysosomes are arranged in a tubular fashion within the macrophage cells of persons with CHS. Affected neutrophils and monocytes have a chemotactic and migratory capacity that is about 40% that of normal cells. Affected patients also have a tendency to bleeding due to platelet dysfunction. As a result of decreased responsiveness to chemotactic stimuli, the malfunctioning immune system cannot protect the body from severe, recurrent infections. CHS is often fatal because patients are immunocompromised and are incapable of mounting an effective immune response to infection.

In the Beige (Bg) mouse strain the mouse homologue of LYST (“Lyst”) is disrupted by a deletion that precludes the expression of functional Lyst protein. Bg mice were found to exhibit reduced proliferative diseases, including a lower rate of stenosis following implantation of vascular tissue grafts, as compared to wild-type mice. The effect of the dysfunctional Lyst protein in Bg mice is highly macrophage-specific, as determined by bone marrow transplant experiments (see Examples).

C. Inhibitors of LYST

It has been established that blockade of the LYST protein can reduce or prevent immune processes that give rise to vascular proliferative disorders, fibroproliferative disease and excessive scar tissue following injury Immuno-modulatory agents that inhibit or reduce the transcription, translation or function of the LYST protein are disclosed.

Inhibitors of LYST can bind to the LYST gene or to LYST protein and directly or indirectly block the biological function of LYST protein. Inhibitors can also block the biological function of one or more signaling pathways that constitute the down-stream biological function of LYST. In some embodiments, inhibitors of LYST act by preventing endogenous ligands of the LYST protein from interacting with or binding directly to the LYST protein. The inhibitors can block protein-protein interactions involving the LYST protein, or they can prevent or reduce the functional activity of a complex of the LYST protein and a ligand. Inhibitors that bind directly to the LYST protein may act by direct occlusion of an active site on the LYST protein, or through indirect occlusion, such as by stearic blockade of LYST interactions. For example, in some embodiments the inhibitor obstructs or occludes the function of a protein interaction domain, such as the solenoid protein domain formed by tandem copies of the WD40 repeat motif. In other embodiments, inhibitors bind to a location that is spatially distinct from an active site.

Inhibitors that bind to the LYST protein can prevent LYST function by mechanisms including, but not limited to, inducing dimerization, inducing oligomerization, inducing conformational changes, preventing catalytic functions, inducing degradation, inducing uptake by immune cells, preventing uptake by target cells, preventing ligand binding, preventing phosphorylation, inducing denaturation, preventing one or more post-translational modifications or otherwise altering the native tertiary structure of the LYST protein.

It is understood that initiation or transduction of cellular signaling pathways by LYST can require binding of a ligand to the LYST protein. Therefore, proteins, antibodies or small molecules that block signal transduction pathways involving LYST and optionally prevent co-ligation of LYST and its receptors are useful immune-modulatory agents. Classes of LYST inhibitors discussed below include antibodies and functional nucleic acids.

1. Antibodies

Antibodies that inhibit the function of LYST by binding directly to the LYST protein, its ligands or its accessory molecules are disclosed. Any specific antibody can be used in the methods and compositions provided herein. Antibodies can include an antigen binding site that binds to an epitope on the LYST protein. Binding of an antibody to LYST can inhibit or reduce the function of the LYST protein via one or more distinct mechanisms.

In some embodiments, the antibody or antigen binding fragment binds specifically to an epitope within the protein encoded by the amino acid sequence of SEQ ID NO: 2. The epitope can be a linear epitope and can include one or more consecutive amino acids of the primary sequence of SEQ ID NO: 2. In other embodiments, the antibody or antigen binding fragment thereof can bind a conformational epitope that includes a 3-D surface feature, shape, or tertiary structure of the LYST protein. In some embodiments, a 3-D surface feature can include any number of amino acids from SEQ ID NO: 2, or the corresponding residues in a homolog, ortholog, paralog, or variant thereof.

In some embodiments, the antibody or antigen binding fragment that binds specifically to an epitope within the protein encoded by the amino acid sequence of SEQ ID NO: 2 can only bind if the protein encoded by the amino acid sequence of SEQ ID NO: 2 is not bound by a ligand or small molecule.

Various types of antibodies and antibody fragments can be used in the disclosed compositions and methods, including whole immunoglobulin of any class, fragments thereof, and synthetic proteins containing at least the antigen binding variable domain of an antibody. The antibody can be an IgG antibody, such as IgG₁, IgG₂, IgG₃, or IgG₄. An antibody can be in the form of an antigen binding fragment including a Fab fragment, F(ab′)2 fragment, a single chain variable region, and the like. Antibodies can be polyclonal or monoclonal (mAb). Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity (U.S. Pat. No. 4,816,567; and Morrison, et al., Proc. Natl. Acad. Sci. USA, 81: 6851-6855 (1984)). The disclosed antibodies can also be modified by recombinant means, for example by deletions, additions or substitutions of amino acids, to increase efficacy of the antibody in mediating the desired function. Substitutions can be conservative substitutions. For example, at least one amino acid in the constant region of the antibody can be replaced with a different residue (see, e.g., U.S. Pat. No. 5,624,821; U.S. Pat. No. 6,194,551; WO 9958572; and Angal, et al., Mol. Immunol. 30:105-08 (1993)). In some cases changes are made to reduce undesired activities, e.g., complement-dependent cytotoxicity. The antibody can be a bi-specific antibody having binding specificities for at least two different antigenic epitopes. In one embodiment, the epitopes are from the same antigen. In another embodiment, the epitopes are from two different antigens. Bi-specific antibodies can include bi-specific antibody fragments (see, e.g., Hollinger, et al., Proc. Natl. Acad. Sci. U.S.A., 90:6444-48 (1993); Gruber, et al., J. Immunol., 152:5368 (1994)).

A variety of antibodies that bind the human LYST protein are commercially available from multiple sources, for example, Santa Cruz Biotechnology, CA, USA, Cat. No. sc-136746). Antibodies can be generated by any means known in the art. Exemplary descriptions means for antibody generation and production include Delves, Antibody Production: Essential

Techniques (Wiley, 1997); Shephard, et al., Monoclonal Antibodies (Oxford University Press, 2000); Goding, Monoclonal Antibodies: Principles And Practice (Academic Press, 1993); and Current Protocols In Immunology (John Wiley & Sons, most recent edition). Fragments of intact Ig molecules can be generated using methods well known in the art, including enzymatic digestion and recombinant means.

2. Functional Nucleic Acids

Functional nucleic acids that inhibit the transcription, translation or function of LYST gene products are disclosed. Functional nucleic acids are nucleic acid molecules that have a specific function, such as binding a target molecule or catalyzing a specific reaction. As discussed in more detail below, functional nucleic acid molecules can be divided into the following non-limiting categories: antisense molecules, siRNA, miRNA, aptamers, ribozymes, triplex forming molecules, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains.

Thus, functional nucleic acids can interact with the mRNA or the genomic DNA of the LYST polypeptide or they can interact with the LYST polypeptide itself. Functional nucleic acids are often designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place. Therefore the disclosed compositions can include one or more functional nucleic acids designed to reduce expression or function of the LYST protein.

In some embodiments, the composition includes a functional nucleic acid or polypeptide designed to target and reduce or inhibit expression or translation of LYST mRNA; or to reduce or inhibit expression, reduce activity, or increase degradation of LYST protein. In some embodiments, the composition includes a vector suitable for in vivo expression of the functional nucleic acid.

In some embodiments, a functional nucleic acid or polypeptide is designed to target a segment of the nucleic acid encoding the amino acid sequence of SEQ ID NO: 2, or the complement thereof, or variants thereof having a nucleic acid sequence 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a nucleic acid encoding the amino acid sequence of SEQ ID NO: 2.

In other embodiments, a functional nucleic acid or polypeptide is designed to target a segment of the nucleic acid sequence of SEQ ID NO: 1, or the complement thereof, or variants thereof having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1

In some embodiments, the function nucleic acid hybridizes to the nucleic acid of SEQ ID NO: 1, or a complement thereof, for example, under stringent conditions. In some embodiments, the function nucleic acid hybridizes to a nucleic acid sequence that encodes SEQ ID NO: 2, or a complement thereof, for example, under stringent conditions.

Methods of making and using vectors for in vivo expression of the disclosed functional nucleic acids such as antisense oligonucleotides, siRNA, shRNA, miRNA, EGSs, ribozymes, and aptamers are known in the art.

i. Antisense Molecules

The functional nucleic acids can be antisense molecules. Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. Exemplary methods include in vitro selection experiments and DNA modification studies using DMS and DEPC. It is preferred that antisense molecules bind the LYST target molecule with a dissociation constant (K_{d}) less than or equal to 10⁻⁶, 10⁻⁸, 10⁻¹⁰, or 10⁻¹².

ii. Aptamers

The functional nucleic acids can be aptamers. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 10⁻¹² M. It is preferred that the aptamers bind the LYST target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a K_{d} with the LYST target molecule at least 10, 100, 1000, 10,000, or 100,000 fold lower than the K_{d} with a background binding molecule. It is preferred when doing the comparison for a molecule such as a polypeptide, that the background molecule be a different polypeptide.

iii. Ribozymes

The functional nucleic acids can be ribozymes. Ribozymes are nucleic acid molecules that are capable of catalyzing a chemical reaction, either intra-molecularly or inter-molecularly. It is preferred that the ribozymes catalyze intermolecular reactions. Different types of ribozymes that catalyze nuclease or nucleic acid polymerase-type reactions which are based on ribozymes found in natural systems, such as hammerhead ribozymes are disclosed. Ribozymes that are not found in natural systems, but which have been engineered to catalyze specific reactions de novo are also disclosed. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for targeting specific cleavage of nucleic acids because recognition of the target substrate is based on the target substrates sequence.

iv. Triplex Forming Oligonucleotides

The functional nucleic acids can be triplex forming oligonucleotide molecules. Triplex forming functional nucleic acid molecules are molecules that can interact with either double-stranded or single-stranded nucleic acid. When triplex molecules interact with a target region, a structure called a triplex is formed in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹².

v. External Guide Sequences

The functional nucleic acids can be external guide sequences. External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukaryotic cells. Representative examples of how to make and use EGS molecules to facilitate cleavage of a variety of different target molecules are known in the art.

vi. RNA Interference

In some embodiments, the functional nucleic acids induce gene silencing through RNA interference (siRNA). Expression of the LYST gene can be effectively silenced in a highly specific manner through RNA interference.

Gene silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III-like enzyme called Dicer, into double stranded small interfering RNAs (siRNA) 21-23 nucleotides in length that contain 2 nucleotide overhangs on the 3′ ends (Elbashir, et al., Genes Dev., 15:188-200 (2001); Bernstein, et al., Nature, 409:363-6 (2001); Hammond, et al., Nature, 404:293-6 (2000); Nykanen, et al., Cell, 107:309-21 (2001); Martinez, et al., Cell, 110:563-74 (2002)). The effect of iRNA or siRNA or their use is not limited to any type of mechanism

In one embodiment, a siRNA triggers the specific degradation of homologous LYST RNA molecules, such as LYST mRNAs, within the region of sequence identity between both the siRNA and the target LYST RNA.

Sequence specific gene silencing can be achieved in mammalian cells using synthetic, short double-stranded RNAs that mimic the siRNAs produced by the enzyme dicer (Elbashir, et al., Nature, 411:494-498 (2001)) (Ui-Tei, et al., FEBS Lett, 479:79-82 (2000)).

siRNA can be chemically or in vitro-synthesized or can be the result of short double-stranded hairpin-like RNAs (shRNAs) that are processed into siRNAs inside the cell. For example, WO 02/44321 discloses siRNAs capable of sequence-specific degradation of target mRNAs when base-paired with 3′ overhanging ends, herein incorporated by reference for the method of making these siRNAs. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit. In some embodiments, the composition includes a vector expressing the functional nucleic acid. The production of siRNA from a vector is more commonly done through the transcription of a short hairpin RNAse (shRNAs). Kits for the production of vectors including shRNA are available, such as, for example, Imgenex's GENESUPPRESSOR™ Construction Kits and Invitrogen's BLOCK-IT™ inducible RNAi plasmid and lentivirus vectors. In some embodiments, the functional nucleic acid is siRNA, shRNA, or miRNA.

B. Excipients, Delivery Vehicles and Devices

LYST inhibitors can be administered and taken up into the cells of a subject with or without the aid of a delivery vehicle. Appropriate delivery vehicles for the disclosed inhibitors are known in the art and can be selected to suit the particular inhibitor. In a preferred embodiment, the inhibitor is delivered by injection intravenously, subcutaneously, intraperitoneally, or locally. Typical carriers are saline, phosphate buffered saline, and other injectable carriers.

Formulations including one or more LYST inhibitors with or without delivery vehicles are disclosed. The disclosed LYST inhibitors can be formulated into pharmaceutical compositions including one or more pharmaceutically acceptable carriers. Pharmaceutical compositions can be formulated for different mechanisms of administration, according to the inhibitor and the intended use. Pharmaceutical compositions formulated for administration by parenteral (intramuscular, intraperitoneal, intravenous (IV) or subcutaneous injection), topical or transdermal (either passively or using iontophoresis or electroporation) routes of administration or using bioerodible inserts are disclosed.

1. Parenteral Administration

In some embodiments, one or more LYST inhibitors and optionally a delivery vehicle are formulated for administration in an aqueous solution, by parenteral injection. The formulation may also be in the form of a suspension or emulsion. In general, pharmaceutical compositions are provided including effective amounts of an active agent, targeting moiety, and optional a delivery vehicle and optionally include pharmaceutically acceptable diluents, preservatives, solubilizers, emulsifiers, adjuvants and/or carriers. Such compositions include the diluents sterile water, buffered saline of various buffer content (e.g., Tris-HCl, acetate, phosphate), pH and ionic strength and optionally additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Pulmonary and Mucosal Administration

In further embodiments, one or more LYST inhibitors and optionally a delivery vehicle are formulated for administration to the mucosa, such as the mouth, eyes, lungs, nasal, oral (sublingual, buccal), vaginal, or rectal mucosa.

Formulations for administration to the mucosa will typically be spray dried drug particles, which may be incorporated into a tablet, gel, capsule, suspension or emulsion. Standard pharmaceutical excipients are available from any formulator.

In one embodiment, the compounds are formulated for pulmonary delivery, such as intranasal administration or oral inhalation. The respiratory tract is the structure involved in the exchange of gases between the atmosphere and the blood stream. The upper and lower airways are called the conducting airways. The terminal bronchioli divide into respiratory bronchiole, which then lead to the ultimate respiratory zone, the alveoli, or deep lung. The deep lung, or alveoli, is the primary target of inhaled therapeutic aerosols for systemic drug delivery. Therapeutic agents that are active in the lungs can be administered systemically and targeted via pulmonary absorption. The term aerosol as used herein refers to any preparation of a fine mist of particles, which can be in solution or a suspension, whether or not it is produced using a propellant. Aerosols can be produced using standard techniques, such as ultra-sonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

Compositions can be delivered to the lungs while inhaling and traverse across the lung epithelial lining to the blood stream when delivered either as an aerosol or spray dried particles having an aerodynamic diameter of less than about 5 microns.

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits. Formulations for pulmonary delivery include unilamellar phospholipid vesicles, liposomes, or lipoprotein particles. Formulations and methods of making such formulations containing nucleic acid are well known to one of ordinary skill in the art. A wide range of mechanical devices designed for pulmonary delivery of therapeutic products can be used, including but not limited to nebulizers, metered dose inhalers, and powder inhalers, all of which are familiar to those skilled in the art.

3. Topical and Transdermal Administration

The active agent and optional delivery vehicle can be applied topically. Topical administration can include application directly to the vasculature or to tissues or prostheses, for example during surgery, or by direct administration to the skin.

Transdermal formulations may also be prepared. These will typically be ointments, lotions, sprays, or patches, all of which can be prepared using standard technology. Transdermal formulations can include penetration enhancers. Standard pharmaceutical excipients for topical or transdermal administration are available from any formulator.

4. Controlled Delivery Matrices

Controlled release polymeric devices can be made for long term release systemically following implantation of a polymeric device (rod, cylinder, film, disk) or injection (microparticles). The matrix can be in the form of microparticles such as microspheres, where LYST inhibitors are dispersed within a solid polymeric matrix or microcapsules, where the core is of a different material than the polymeric shell, and one or more LYST inhibitor(s) is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of disclosed inhibitors of LYST, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be cross-linked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release 5:13-22 (1987); Mathiowitz, et al., Reactive Polymers 6:275-283 (1987); and Mathiowitz, et al., J. Appl. Polymer Sci. 35:755-774 (1988). The devices can be formulated for local release to treat the area of implantation or injection, which will typically deliver a dosage that is much less than the dosage for treatment of an entire body. The devices can also be formulated for local or systemic delivery.

Micro and nanoparticles designed to deliver cargo such as drugs and antibodies to the vasculature, to vascular smooth muscle cells, or sites or clots or thrombosis are known in the art. See, for example, Wickline, et al., Arteriosclerosis, Thrombosis, and Vascular Biology, 26:435-441 (2006), published online (December 2005), and U.S. Published Application Nos. 2002/0168320, 2003/0086867, 2003/0129136, 2004/0058951, 2004/0115192, 2006/0147380, 2006/0239919, 2007/0140965, 2007/0202040, 2007/0258908, 2008/0175792, 2008/0247943, and 2013/0064765.

For example, perfluorocarbon nanoparticles, previously considered as artificial blood substitutes, have been developed into a platform technology for molecular imaging and targeted drug delivery, i.e., a so-called “theranostic” technology. These lipid-encapsulated particles, which are nominally 250 nm in diameter, can be administered intravenously and are typically constrained by size to the intact vasculature.

In some embodiments the delivery vehicle is a liposome. If the LYST inhibitor is an antibody, protein or small molecule, the preferred delivery vehicle can be a liposome. Liposomes are disclosed for the delivery of the disclosed LYST inhibitors directly to a certain cell type, for example macrophage cells. Macrophage cells can internalize liposomes, leading to the delivery of the one or more inhibitors to the intracellular compartments of the macrophage. Suitable methods, materials and lipids for making liposomes are known in the art. Liposome delivery vehicles are commercially available from multiple sources. Liposomes can be formed from a single lipid bilayer (i.e., the liposome can be unilamellar) or several concentric lipid bilayers (i.e., the liposome can be multilamellar). The liposome may be formed from a single lipid; however, in some embodiments, the liposome is formed from a combination of more than one lipid. The lipids can be neutral, anionic or cationic at physiologic pH.

Suitable neutral and anionic lipids include sterols and lipids such as cholesterol, phospholipids, lysolipids, lysophospholipids, and sphingolipids. Neutral and anionic lipids include, but are not limited to, phosphatidylcholine (PC) (such as egg PC, soy PC), including 1,2-diacyl-glycero-3-phosphocholines; phosphatidylserine (PS), phosphatidylglycerol, phosphatidylinositol (PI); glycolipids; sphingophospholipids, such as sphingomyelin, sphingoglycolipids (also known as 1-ceramidyl glucosides), such as ceramide galactopyranoside, gangliosides and cerebrosides; fatty acids, sterols containing a carboxylic acid group such as cholesterol or derivatives thereof; and 1,2-diacyl-sn-glycero-3-phosphoethanolamines, including 1,2-dioleoyl-sn-Glycero-3-phosphoethanolamine or 1,2-dioleolylglyceryl phosphatidylethanolamine (DOPE), 1,2-dihexadecylphosphoethanolamine (DHPE), 1,2-distearoylphosphatidylcholine (DSPC), 1,2-dipalmitoylphosphatidylcholine (DPPC), and 1,2-dimyristoylphosphatidylcholine (DMPC). Trimethyl ammonium salts, also referred to as TAP lipids, for example as a methylsulfate salt. Suitable TAP lipids include, but are not limited to,

DOTAP (dioleoyl-), DMTAP (dimyristoyl-), DPTAP (dipalmitoyl-), and DSTAP (distearoyl-). Other suitable cationic lipids include dimethyldioctadecyl ammonium bromide (DDAB), 1,2-diacyloxy-3-trimethylammonium propanes, N-[1-(2,3-dioloyloxy)propyl]-N,N-dimethyl amine (DODAP), Other suitable lipids include PEGylated derivatives of the neutral, anionic, and cationic lipids described above. Incorporation of one or more PEGylated lipid derivatives can result in a liposome which displays polyethylene glycol chains on its surface. The resulting liposomes may possess increased stability and circulation time in vivo as compared to liposomes lacking PEG chains on their surfaces.

If the LYST inhibitor is a nucleic acid or vector, the delivery vehicle can be a viral vector, for example, a commercially available preparation, such as an adenovirus vector (Quantum Biotechnologies, Inc. (Laval, Quebec, Canada). The viral vector delivery can be via a viral system, such as a retroviral vector system which can package a recombinant retroviral genome (see e.g., Pastan et al., (1988) Proc. Natl. Acad. Sci. U.S.A. 85:4486; Miller et al., (1986) Mol. Cell. Biol. 6:2895). The recombinant retrovirus can then be used to infect and thereby deliver to the infected cells nucleic acid encoding the LYST inhibitor. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948 (1994)), adeno-associated viral (AAV) vectors (Goodman et al., Blood 84:1492-1500 (1994)), lentiviral vectors (Naidini et al., Science 272:263-267 (1996)), pseudotyped retroviral vectors (Agrawal et al., Exper. Hematol. 24:738-747 (1996)).

Physical transduction techniques can also be used, such as liposome delivery and receptor-mediated and other endocytosis mechanisms (see, for example, Schwartzenberger et al., Blood 87:472-478 (1996)). Commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art are well known. In addition, the disclosed nucleic acid or vector can be delivered in vivo by electroporation, the technology for which is available from Genetronics, Inc. (San Diego, Calif.) as well as by means of a sonoporation or sonication machine (ImaRx Pharmaceutical Corp., Tucson, Ariz.). The disclosed compositions and methods can be used in conjunction with any of these or other commonly used gene transfer methods.

5. Grafts and Medical Devices

The disclosed compositions for immunomodulation by inhibition of LYST can be coated onto or incorporated into medical devices, or can be used to pre-treat implantable vascular grafts ex vivo.

Grafts

LYST inhibitors can be used to pre-treat vascular grafts ex vivo prior to implantation in a subject. Compositions including one or more LYST inhibitors and optionally a delivery vehicle can be applied to the tissue by methods to insure that they adhere and are distributed throughout the tissue or graft in optimal locations for drug treatment. In some embodiments, one or more LYST inhibitor(s) is delivered using nanoparticles, microparticles, liposomes, micelles, emulsions, gels or coatings that are designed to adhere to the regions of interest, to carry sufficient drug load to provide local treatment for prolonged periods, to release the LYST inhibitors at a desired rate or schedule, to penetrate into tissue or graft to an optimal extent, or a combination thereof.

Grafts for use in surgery can also be formed by seeding cells ex-vivo onto a biodegradable scaffold. The grafts can be autologous, for example, saphenous vein or radial artery; preserved autologous, for example, cryopreserved vein; allogeneic; xenogenic; or synthetic, for example, woven polyester, polyurethane (LYCRA®), polytetraflouroethylene (PTFE), GORE-TEX®, or polyethelene terephthalate (DACRON®). Arterial homografts using internal mammary, radial or hypogastric arteries are examples of useful and durable vascular conduits. Exemplary grafts are discussed below.

Tissue Engineering Vascular Graft

Tissue engineering vascular grafts (TEVG) including one or more inhibitors of LYST are disclosed. Tissue-engineered vascular grafts (TEVGs) hold great promise for advancing the field of congenital heart surgery, where their growth capacity can be used to its fullest potential. For example, TEVGs can be designed for use as a vascular conduit connecting the inferior vena cava to the pulmonary artery in patients undergoing modified Fontan surgery. Long-term (>10 years) results of a pilot study evaluating the use of the TEVG in 25 patients with single ventricle cardiac anomalies demonstrated no graft related deaths or graft failures. It also confirmed the growth capacity of the TEVG making it the first man made graft with growth capacity. The primary graft-related complication was stenosis, effecting approximately 30% of patients with 16% (4/25) requiring angioplasty to treat critical stenosis.

In one embodiment, a TEVG is formed from a biodegradable tubular scaffold fabricated from a polyglycolic acid-fiber tube. The tube can be coated with a copolymer such as a 50:50 ratio of poly lactic acid (PLA) and poly-caprolactone. The scaffold can be seeded with cells prior to implanting into a subject. In some embodiments the cells are autologous cells from the intended recipient.

It has been established that excessive infiltration of macrophages to tissue engineered vascular grafts (TEVGs) promoted scarring, resulting in vessel thickening, occlusion and stenosis. Therefore, the compositions and methods described herein can be used to deliver one or more LYST inhibitors locally and in a controlled fashion to a TEVG to prevent or inhibit infiltration by macrophages.

Bypass Graft

Bypass grafts including one or more inhibitors of LYST are disclosed. A common form of bypass surgery involves resecting the saphenous vein from the leg for auto-transplantation to the coronary artery. In a significant number of cases these grafts fail, largely due to restenosis caused by neointimal hyperplasia. The compositions and methods described herein can be used to deliver one or more LYST inhibitors locally and in a controlled fashion, to the autologous graft. The inhibitor can be administered prior to, at the time of and/or immediately following surgery. After resection of the saphenous vein the tissue can be (and is often) for hours suspended in saline during chest opening and preparation for graft implantation. Compositions including one or more LYST inhibitors and optional a targeting signal, delivery vehicle or combination thereof can be incubated with the saphenous vein during this time period.

Arteriovenous Graft

Arteriovenous grafts including one or more inhibitors of LYST are disclosed. End stage renal disease is increasing in the United States. Morbidity of hemodialysis access remains a major quality of life issue for patients; it also represents a significant cost to society. A native arteriovenous fistula (AVF) remains the conduit of choice to provide access for hemodialysis and provides superior results when compared with other options such as a prosthetic AVG. Unfortunately each individual is limited in the number of native AVF that can be created due to the limited number of suitable sites and vessels. Access sites are limited as patients with end stage renal disease usually have severe comorbidity, requiring extensive venipuncture for diagnosis and therapy for life. In patients who have exhausted all options for primary AVF, new access sites must use AV grafts. These grafts are susceptible to restenosis by neointimal hyperplasia limiting their effectiveness. Incubation of a composition including one or more LYST inhibitors, and optionally including a targeting signal, a delivery vehicle, or a combination thereof onto the graft can be done at the time of surgery.

Medical Devices

In some embodiments, a composition including one or more LYST inhibitors is coated onto, or incorporated into, a medical device or component thereof (such as into polymeric reservoirs in vascular stents) to reduce or inhibit vascular proliferation disorders such as neointima formation in a subject. The device can be a device that is inserted into the subject transiently, or a device that is implanted permanently. In some embodiments, the device is a surgical device.

Examples of medical devices include, but are not limited to, needles, cannulas, catheters, shunts, balloons, and implants such as stents and valves.

In some embodiments, the LYST inhibitor or pharmaceutical composition can be formulated to permit its incorporation onto the medical device, which can apply the inhibitor directly to the site to prevent or treat conditions such restenosis or other vascular proliferation disorders. The LYST inhibitor or pharmaceutical composition thereof can be formulated by including it within a coating on the medical device. There are various coatings that can be utilized such as, for example, polymer coatings that can release the inhibitor over a prescribed time period. The inhibitor, or a pharmaceutical composition thereof, can be embedded directly within the medical device. In some embodiments, the LYST inhibitor is coated onto or within the device in a delivery vehicle such as a microparticle or liposome that facilitates its release and delivery.

Stents

In some embodiments, the medical device is a vascular implant such as a stent. Stents are utilized in medicine to prevent or eliminate vascular restrictions. Stents can be inserted into a restricted vessel so that the restricted vessel is widened. The experience with such vascular implants indicates that excessive growth of the adjacent cells results again in a restriction of the vessel particularly at the ends of the implants which results in reduced effectiveness of the implants. If a stent is inserted into a human artery for the elimination of an arteriosclerotic stenosis, intimal hyperplasia can occur within a year at the ends of the vascular implant and results in renewed stenosis.

Accordingly, in some embodiments, the stents are coated or loaded with a composition including a LYST inhibitor and optionally a delivery vehicle.

Many stents are commercially available or otherwise know in the art. Stents can be formed, i.e., etched or cut, from a thin tube of suitable material, or from a thin plate of suitable material and rolled into a tube.

Suitable materials for the stent include but are not limited to stainless steel, iridium, platinum, gold, tungsten, tantalum, palladium, silver, niobium, zirconium, aluminum, copper, indium, ruthenium, molybdenum, niobium, tin, cobalt, nickel, zinc, iron, gallium, manganese, chromium, titanium, aluminum, vanadium, and carbon, as well as combinations, alloys, and/or laminations thereof. For example, the stent may be formed from a cobalt alloy, such as L605 or MP35N®, Nitinol (nickel-titanium shape memory alloy), ABI (palladium-silver alloy), Elgiloy® (cobalt-chromium-nickel alloy), etc. It is also contemplated that the stent may be formed from two or more materials that are laminated together, such as tantalum that is laminated with MP35N®. The stents may also be formed from wires having concentric layers of different metals, alloys, or other materials. Embodiments of the stent may also be formed from hollow tubes, or tubes that have been filled with other materials. The aforementioned materials and laminations are intended to be examples and are not intended to be limiting in any way.

Stents can be drug-eluting stents. Various drug eluting stents that simultaneously deliver a therapeutic substance to the treatment site while providing artificial radial support to the wall tissue are known in the art. Endo-luminal devices including stents can be coated on their outer surfaces with a substance such as a drug releasing agent, growth factor, antibody, or the like. Stents have also been developed having a hollow tubular structure with holes or ports cut through the sidewall to allow drug elution from a central lumen. Although the hollow nature of the stent allows the central lumen to be loaded with a drug solution that is delivered via the ports or holes in the sidewall of the stent, the hollow tubular structure may not have suitable mechanical strength to provide adequate scaffolding in the vessel.

Stents that elute LYST inhibitors are disclosed. In some embodiments, the devices are also coated or impregnated with a LYST inhibitor and one or more additional therapeutic agents, including, but not limited to, anti-platelet agents, anticoagulant agents, antimicrobial agents, and anti-metabolic agents.

Exemplary stents that can be used with the compositions and methods disclosed herein include, but are not limited to, those described in U.S. Pat. Nos. 5,891,108, 6,918,929, 6,923,828, 6,945,992, 6,986,785, 7,060,090, 7,144,419, 7,163,555, 7,323,008, 7,651,527, 7,655,034, 7,678,141, 7,744,645, 7,942,917, 8,001,925, 8,001,925, 8,034,099, 8,048,149, 8,066,760, 8,100,960, 8,157,855, 8,172,893, 8,182,524, 8,187,284, 8,187,322, 8,197,528, 8,206,432, 8,221,490, 8,231,669, 8,236,044, 8,252,048, 8,252,065, 8,257,425, 8,257,431, 8,292,945, 8,298,278, 8,298,280, 8,348,991, 8,348,992, 8,348,993, 8,353,952, 8,359,998, 8,361,140, 8,372,134, 8,372,138, 8,377,112, 8,388,676, 8,398,695, 8,414,637, 8,414,639, and 8,414,656.

BioProstheses

Multiple other prosthetic devices can include one or more inhibitors of LYST, including but not limited to heart valves, artificial joints, pacemakers, indwelling catheters and cosmetic implants.

It is believed that the disclosed LYST inhibitors can reduce or prevent inflammatory responses and fibroproliferative disorders associated with the presence of a bioprosthesis. Accordingly, in some embodiments, the bioprostheses are coated or loaded with a composition including a LYST inhibitor and optionally a delivery vehicle.

### IV. Methods of Use

Methods of using the disclosed LYST inhibitors are provided. The methods can include administering to a subject an effective amount of a composition including one or more LYST inhibitors to prevent, reduce, or inhibit the expression or function of LYST in the subject; pretreating a medical device or vascular graft with an effective amount of a composition including a LYST inhibitor to prevent, reduce, or inhibit neointima formation following insertion or implantation of the device or graft into the subject; or a combination thereof.

A. Disorders and Diseases to be Treated

LYST inhibitors can be used to alter immune functions, including, but not limited to, macrophage, platelet, and natural killer cell function, creating a pro-regenerative immune environment. Methods of using LYST inhibitors including, but not limited to, methods designed to inhibit or block transcription, translation, or function of the LYST protein can be used to modulate the immune response. In some embodiments, the methods promote vascular neotissue formation, inhibit the formation of neointima and improve vascular patency, for example, following vascular surgery. Methods of using LYST inhibitors for preventing intimal and neointimal hyperplasia in both biological and synthetic vascular conduits are provided. The methods can reduce or prevent TEVG stenosis and improve TEVG function. Additionally, LYST inhibitors can have broader implications for promoting tissue regeneration, improving wound healing, and modulating the foreign body response. Thus, methods of using LYST inhibitors as an adjunct to regenerative medicine applications are also described. In some embodiments, the methods of using LYST inhibitors can promote healing and prevent scar formation, for example, promoting healing and preventing adhesions after abdominal surgery.

The methods can modulate the amount of Platelet Derived Growth Factor (PDGF) produced as a result of tissue injury. Therefore, the methods can treat diseases associated with excessive or undesirable expression of PDGF.

The methods can also prevent or treat diseases resulting from excessive fibrosis, for example fibrotic diseases of the liver, lung or heart. Finally, methods of anti-LYST therapy can result in immune modulation that alters the foreign body reaction and promotes integration but blocks encapsulation of bio-prostheses, thus improving function and longevity of devices such as pacemaker or nerve stimulators or integration of replacement heart valves or artificial joints.

In preferred embodiments, one or more LYST inhibitors are effective to reduce, inhibit, or delay one or more symptoms of a disease, disorder or condition in a subject. The disclosed LYST inhibitors have a wide variety of therapeutic and prophylactic uses, for example, they can be used to treat or prevent vascular proliferative disorders following injury or various surgical procedures, scarring and fibrotic diseases. Methods of using one or more inhibitors of LYST for treating a disease characterized by the presence of excessive scar or fibrotic tissue are described. Methods of using one or more LYST inhibitors for anti-platelet therapy, for example, for treating diseases resulting from aberrant, excessive or otherwise undesirable platelet activity or PDGF signaling are also provided.

Vascular Proliferative Disorders

In some embodiments, the disclosed LYST inhibitors can be used to treat or prevent vascular proliferative disorders. Examples of such disorders include, but are not limited to, vascular proliferation involved in atherosclerosis, vascular proliferation following intravascular device implantation, vascular proliferation at the site of vascular anastamosis as generally occurs following revascularization procedure or A-V shunting, vascular proliferation following carotid endarterectomy, and transplant vasculopathy.

Methods of treating or preventing vascular proliferative disorders by administration of one or more LYST inhibitors are provided. The methods typically reduce or inhibit the infiltration of macrophage cells, or the conversion of macrophage cells from M1 to M2 phenotype, or both, compared to a control. In some embodiments, the methods reduce or inhibit proliferation of macrophage cells without reducing or inhibiting vascular neotissue development. A subject can have stenosis, restenosis or other vascular proliferation disorders, or be identified as being at risk for restenosis or other vascular proliferation disorders, for example subjects who have undergone, are undergoing, or will undergo a vascular trauma, angioplasty, surgery, or transplantation arteriopathy, etc. Diseases, disorders and conditions that can be treated using the disclosed compositions are discussed in more detail below.

Vascular Injury

In some embodiments, one or more LYST inhibitors can be applied before, at the time of or following a vascular trauma.

Vascular injury triggers a cascade of events that includes endothelial denudation or dysfunction, inflammation, as well as activation and proliferation of vascular smooth muscle cells (VSMC). Multiple growth factors and cytokines are released by dysfunctional endothelial cells, inflammatory cells, platelets and VSMCs. These growth factors and cytokines mediate chemo-attraction, cell migration, proliferation, apoptosis and matrix modulation, and are implicated in a number of vascular proliferative disorders.

Vascular proliferative diseases and disorders can be initiated by mechanical, biochemical or immunological injury to blood vessel walls. Typical vascular trauma include those associated with both blunt and penetrating injuries including, but not limited to, lacerations, puncture wounds, crush injuries, gunshot wounds, knife wounds, occupational injuries, falls, and motor vehicle accidents, as well as medical interventions, such as surgery or angioplasty.

In some embodiments, the subject has undergone, is undergoing, or will undergo a surgery. Surgeries can include invasive, minimally invasive, or percutaneous surgery. For example, in some embodiments the subject is having surgery to treat or repair abdominal aortic aneurysm, carotid stenosis, varicose veins, peripheral arterial occlusive disease, acute limb ischemia, or aortic dissection. Common vascular surgeries include, but are not limited to, open abdominal aortic aneurysm repair, endovascular aneurysm repair (EVAR), carotid endarterectomy, carotid stenting, vein stripping, sclerotherapy and foam sclerotherapy, endo-venous laser treatment, radiofrequency vein ablation, ambulatory phlebectomy, angioplasty with/out stenting, bypass surgery endarterectomy atherectomy, balloon embolectomy, thrombectomy, bypass surgery, open repair, thoracic endovascular aneurysm repair (TEVAR). A surgeon can apply one or more LYST inhibitors to the surgical site at the time of surgery, prior to surgery or following surgery, to enhance the process of wound healing, or to prevent the development of vascular proliferative disorders, such as those that give rise to stenosis or restenosis.

Intimal Hyperplasia

Intimal hyperplasia is a physiological healing response to injury of the endothelia of blood vessels. Injury to the endothelial layer triggers a series of acute and chronic inflammatory responses that trigger the aggregation of platelets, the deposition of fibrin and attracts leukocytes to the area (Murakami, et al., Am J Physiol., 272:L197-L202 (1997); Cotran, et al., J Am Soc Nephrol., 1:225-235 (1990)). Thus, regenerative processes that give rise to neovessel formation and intimal hyperplasia appear to be immune-mediated phenomena, similar to the proposed role for monocytes-macrophages in other human vascular biological processes, such as vein graft adaptation (Ratliff and Myles, Arch. Pathol. Lab. Med. 113:772-776 (1989); Motwani and Topol, Circulation 97:916-931 (1998)).

Stenosis

Intimal hyperplasia can lead to thickening of the Tunica intima of a blood vessel, leading to a complication of stenosis of the blood vessel. Activation of inflammatory and pro-coagulant mechanisms is thought to contribute significantly to the initiation and development of stenosis. Over a period of time ranging from a few weeks to months, smooth muscle cells from the medial region of an injured blood vessel relocate to the intimal region. These cells proliferate and deposit extracellular matrix to form a neo-intima at the site of the injury in a process analogous to scar formation (Fingerle, et al., Proc Natl Acad Sci., 86:8412 (1989); Clowes, et al., Circ. Res., 56:139-145 (1985)). Thus, a robust healing response leads to an internal thickening of the vessel wall (intimal hyperplasia) and eventually reduces the vessel lumen, causing stenosis. The formation of intimal hyperplasia can be accelerated by the presence of foreign material such as prosthesis within the vessel, and can result from endovascular intervention including angioplasty, bypass, and transplantation arteriopathy, etc. (Glagov, Circulation, 89:2888-2891 (1994)).

Therefore, methods of using the disclosed LYST inhibitors preventing intimal hyperplasia and stenosis are provided.

### Restenosis

Methods to prevent or reduce restenosis of the coronary vasculature or the peripheral circulatory system are provided. Restenosis of blood vessels is typically due to intimal hyperplasia. A surgical device, such as a stent, may be inserted to open the stenosed vessel, however this is also problematic because the stent itself can stimulate further intimal hyperplasia. In addition, hyperplastic intimal tissue can grow through the interstices of a bare stent and re-stenose the vessel. Whilst covered stents may prevent this from happening, intimal hyperplasia can still occur at the ends of the stent where there is most irritation of the vessel wall. Patients with in-stent restenosis are at risk of serious complications, as stenosis from intimal hyperplasia is often difficult to treat. Unlike soft atheromatous plaques, these stenoses are firm and require prolonged high inflation pressures to dilate with a balloon. The stenoses often recur and repeated dilatation of the vessel leads to repeated intimal injury and perpetuates the intimal healing response.

Accordingly, the disclosed compositions, devices, or grafts can be administered to a subject to reduce or inhibit smooth muscle cell proliferation, migration, and a combination thereof in an amount effective to reduce or inhibit neointima formation and thereby treat or prevent restenosis and other vascular proliferation disorders in the subject. In some embodiments, the patency of grafts and devices can be increased using a composition containing a LYST inhibitor. Therefore, methods for administering a composition containing a LYST inhibitor to devices and grafts or to the subject prior to or after implantation are provided.

Atherosclerosis

Atherosclerosis involves multiple processes, including inflammation, vascular proliferation and matrix alteration (reviewed in Dzau, et al., Nat Med., 8(11) (2002)). In atherosclerosis, VSMC give rise to inflammation, the retention of lipoproteins from the blood, as well as the development of a fibrous deposit that constitute a plaque. Inflammation has been shown to mediate all stages of atherosclerosis: in the development of an atheroma plaque, VSMCs produce pro-inflammatory mediators such as monocyte chemo-attractant proteins, and synthesize matrix molecules that give rise to the retention of lipoproteins from the blood; and following the development of a plaque, local inflammatory milieu can induce collagenase expression and inhibit expression of proteolytic inhibitors, rendering the fibrous cap weak and susceptible to rupture. Therefore, in some embodiments, the disclosed LYST inhibitors are used to treat, reduce, inhibit, or prevent vascular proliferation disorders in a subject.

Angioplasty

In some embodiments, the subject has undergone, is undergoing, or will undergo angioplasty. Angioplasty is the technique of mechanically widening narrowed or obstructed arteries, such as those obstructed as a result of atherosclerosis. Generally, angioplasty includes inserting into a subject's vasculature an empty and collapsed balloon on a guide wire, known as a balloon catheter, which is passed into the narrowed locations and then inflated to a fixed size. The balloon forces expansion of the inner white blood cell/clot plaque deposits and the surrounding muscular wall, opening up the blood vessel for improved flow, and the balloon is then deflated and withdrawn. A stent may or may not be inserted at the time of ballooning to ensure the vessel remains open. Angioplasty includes peripheral angioplasty (i.e., blood vessels outside the coronary arteries, such as in the abdomen, or legs), coronary angioplasty, renal artery angioplasty, carotid angioplasty, and cerebral arteries angioplasty.

In some embodiments, the subject has undergone, is undergoing, or will undergo percutaneous transluminal coronary angioplasty (PTCA). The use of PTCA has greatly reduced the number of fatalities in patients who suffer myocardial infarction (Fischman, et al., N Engl J Med., 331:496-501 (1994); Elezi, et al., Circulation 98:1875-1880 (1998); Bennett and O'Sullivan, Pharmacol Ther., 91:149-166 (2001)). During PTCA, the artery walls are expanded by several times their original diameter in an attempt to increase lumen diameter and improve flow. Unfortunately, this technique is plagued by a high incidence of vessel re-narrowing or restenosis, occurring in 30-40% of patients within 6 months of the procedure (Anderson et al., J Interv. Cardiol., 6:187-202 (1993); Fischman et al., N Engl J Med, 331:496-501 (1994); Elezi et al., Circulation 98:1875-1880 (1998); Bennett and O'Sullivan, Pharmacol Ther, 91:149-166 (2001); Heckenkamp et al., J Cardiovasc. Surg. (Torino), 43:349-357 (2002)).

Prevention of restenosis after successful PTCA remains one of the most challenging tasks in the treatment of obstructive coronary artery disease. Attempts to ameliorate this proliferative response involve the use coronary stents, which have significantly improved both short term and long term outcome following interventional coronary revascularization procedures. Despite a reduction in restenosis rate with stent deployment, restenosis still occurs in 15-30% of patients within 6 months (Fischman et al., N Engl J Med, 331:496-501 (1994); Elezi et al., Circulation, 98:1875-1880 (1998)). This incidence of in-stent restenosis is expected to increase as coronary stenting is becoming more frequent and is used in less ideal lesions. The disclosed LYST inhibitors can be used to treat or prevent restenosis, abdominal adhesions and scarring following angioplasty.

Transplant Arteriopathy

In some embodiments, the subject has undergone, is undergoing, or will undergo a transplant. Chronic transplant arteriopathy (CTA) is a major cause of late allograft loss after heart or kidney transplantation (Taylor, et al., J. Heart Lung Transplant., 24:945-955 (2005), Burke, et al., Transplantation, 60:1413-1417 (1995); Cornell and Colvin, Curr. Opin. Nephrol Hypertens., 14:229-234 (2005)). Therefore, in some embodiments, the disclosed LYST inhibitors are used to reduce, inhibit, or prevent transplant arteriopathy in a transplant recipient.

Excessive Scarring

The disclosed LYST inhibitors can be used to treat, retard or prevent scarring, including the formation of keloids and abdominal adhesions, for example, following injury, disease or surgical procedures.

Methods of using one or more LYST inhibitors to decrease the amount of blood vessel growth at the site of an injury or surgery are provided. In some embodiments the methods prevent or decrease the formation of high density cellular and connective tissue that give rise to scarring, keloids or adhesions. Preferably, the amount of one or more LYST inhibitors does not prevent wound healing.

Hypertrophic Scarring

In some embodiments, the disclosed LYST inhibitors are used to treat, reduce, inhibit, or prevent development of hypertrophic scars. Excessive scarring can occur when the tissue response is out of proportion with the amount of scar tissue required for normal repair and healing. When a deep wound involves significant loss of dermis, hypertrophic scar tissue can be deposited at the wound site. The scar tissue contains a high density of cells, increased volume of connective tissue and an increased vascular supply resulting from increased number of blood vessels. Hypertrophic scars can occur as a result of defects in the remodeling phase of tissue repair (Ehrlich and Kelley, Plast Reconstr Surg., 90:993-998 (1992)).

The disclosed LYST inhibitors can be used to diminish or prevent the development of blood vessels in the area of a wound to reduce, retard or prevent the development of hypertrophic scarring.

Keloids

In some embodiments, the disclosed LYST inhibitors are used to treat, reduce, inhibit, or prevent development of Keloid scars. A keloid scar is a raised or thickened scar that exceeds the boundary of the injury and can continue to develop and enlarge over a prolonged period of time. During development of a keloid growth, collagen, used in wound repair overgrows producing a lump many times larger than that of the original scar.

LYST inhibitors can be used to prevent Keloid development associated with skin injuries, for example, ear piercing, laceration, burns, vaccination or inflammatory processes. LYST inhibitors can be applied locally or topically as needed.

Adhesions

Adhesions are fibrous bands that form between tissues and organs, often as a result of injury during surgery. They may be thought of as internal scar tissues that connect tissues not normally connected, usually across a virtual space such as the peritoneal cavity. Following abdominal surgery or trauma, the production or activity of fibrinolytic enzymes can be compromised because of injury, and fibrinous adhesion develops. If this is allowed to happen, tissue repair cells such as macrophages, fibroblasts and other blood vessel cells penetrate the fibrinous adhesion to deposit collagen and other matrix substances to form a permanent fibrous adhesion.

Exemplary adhesions that can be treated, reduced or prevented by the disclosed LYST inhibitors include abdominal adhesions, pelvic adhesions, including pelvic adhesions that result from endometriosis, pericardial adhesions, peridural adhesions and peritendinous adhesions. Symptoms of adhesions can include abdominal pain, blockages, chronic pelvic pain, cramping, nausea, limited flexibility and inflammation or swelling at the site of the adhesion.

Other Fibroproliferative Diseases

In some embodiments, one or more LYST inhibitors can be applied to treat a fibroproliferative disease.

Fibrosis is the deposition of excess fibrous connective tissue in an organ or tissue in response to inflammation and/or damage (Wynn, Nat Rev Immunol., 4(8): 583-594 (2004)). The repair of damaged tissues involves the production of extracellular matrix components at the site of tissue injury. Macrophages and damaged tissues release cytokines and TGF beta which stimulate cells to lay down connective tissue, including collagen and glycosaminoglycans. However, dysregulation of this process can lead to excessive deposition of this connective tissue, which can obliterate the structure and function of underlying organs, leading to the pathology of fibrotic disease. Fibrosis can occur in many tissues within the body, including the liver, lungs, heart and kidney. Methods of treating major-organ fibrosis and fibroproliferative disorders are provided.

Liver Cirrhosis

Fibrosis of the liver leads to liver damage as hepatocytes are replaced by non-functional scar tissue in a process known as cirrhosis (Masuoka, et al. Ann NY Acad. Sci., 1281:106-122 (2013)). Liver fibrosis and the resulting cirrhosis represent the final common pathway of virtually all chronic liver diseases and can lead to liver failure, liver cancer, and liver-related death. Liver cirrhosis occurs as scar tissue replaces normal parenchyma. Following acute liver injury (e.g., viral hepatitis), parenchymal cells regenerate and replace necrotic or apoptotic cells in a process associated with an inflammatory response. If hepatic injury persists, the liver regeneration process eventually fails and hepatocytes are substituted with abundant extracellular matrix, including fibrillar collagen. Advanced fibrosis is characterized by an accumulation of extracellular matrix proteins rich in fibrillar collagens (predominantly collagen I and collagen III) (reviewed in Iredale, J Clin Invest., 117(3):529-548 (2007); Bataller and Brenner, J Clin Invest, 115(2):209-218 (2005)). Inflammatory cells which activate hepatic stellate cells (HSC) to secrete collagen are an important factor in fibrotic liver disease. Increasing monocyte numbers have been associated with disease progression, specifically with the progression from non-cirrhotic to cirrhotic disease.

Methods of treating liver fibrosis in a subject, including administering to the subject an effective amount of one or more LYST inhibitors to reduce, decrease, limit or prevent one or more symptoms of liver disease relative to an untreated control subject are provided. Typical symptoms of liver diseases include, but are not limited to increased abdominal mass, fatigue, abdominal pain, cachexia, jaundice, obstructive syndromes including lymphatic blockage and accumulation of ascites, anemia and back pain (Sun, et al., Clin J. Oncol. Nurs., 12:759-766 (2008)). In some embodiments, one or more LYST inhibitors improve liver function, reduce inflammation, reduce fibrosis, or a combination thereof in a subject with liver disease relative to an untreated control subject.

Pulmonary Fibrosis

Pulmonary fibrosis is characterized by the deposition of excess fibrous tissue in the lungs that causes marked architectural distortion and loss of alveolar spaces, leading to organ failure and ultimately death from respiratory failure. Pulmonary fibrosis involves gradual exchange of normal lung parenchyma with fibrotic tissue, causing irreversible decrease in oxygen diffusion capacity.

Pulmonary fibrosis (also known as cryptogenic fibrosing alveolitis) is associated with interstitial lung diseases (ILD) such as connective tissue diseases or chronic inflammatory disease (e.g., rheumatoid arthritis), infections, idiopathic lung disease and malignancies. Environmental exposure to inhaled toxins, medications and radiation therapy has also been associated with pulmonary fibrosis.

Methods of treating or preventing pulmonary fibrosis in a subject, including administering to the subject an effective amount of one or more LYST inhibitors to reduce, decrease, limit or prevent one or more symptoms of pulmonary fibrosis relative to an untreated control subject are provided. Typical symptoms include chronic dry cough, shortness of breath, fatigue and weakness, chest discomfort, loss of appetite and weight loss. In some embodiments, one or more LYST inhibitors improve respiratory function, reduce inflammation, reduce fibrosis, or a combination thereof in a subject with pulmonary fibrosis relative to an untreated control subject.

Other Fibrotic Diseases

Methods of treating or preventing diseases characterized by excessive fibrosis in a subject, including administering to the subject an effective amount of one or more LYST inhibitors to reduce, decrease, limit or prevent one or more symptoms of excessive fibrosis relative to an untreated control subject are provided. Exemplary disease and disorders that can be treated by the disclosed methods include nephrosclerosis, scleroderma, Sharp's syndrome, Neurofibromatosis, myelofibrosis, systemic sclerosis, Dupuytren's contracture and macular degeneration. In addition, methods for the treatment of scarring and fibrosis associated with surgical complications, chemotherapeutic, or other drug-induced fibrosis, radiation-induced fibrosis, fibrosis resulting from injury and burns are provided.

Diseases Associated With Platelet Function

Methods of treating or preventing diseases characterized by excessive or deleterious platelet activation and/or platelet derived growth factor (PDGF) mediated signaling are provided. Because platelets play a critical role in the blood clotting process, therapies directed against platelets are some of the most important in the battle against vascular diseases of the heart and brain. Inappropriate or excessive activation of platelets can give rise to clot formation within an intact vessel (thrombosis). A clot that breaks free and begins to travel around the body (thromboembolism) can cause vessel obstruction or occlusion, blood stasis, ischemia, stroke and/or death.

It has been established that reduced LYST leads to reduced platelet activation and reduced expression of PDGF (see Example 7). Accordingly, LYST-inhibitors can be used as anti-platelet drugs, to reduce or prevent deleterious platelet aggregation and thrombus formation. Methods of treating or preventing diseases characterized by deleterious or excessive platelet activation in a subject, including administering to the subject an effective amount of one or more LYST inhibitors to reduce, decrease, limit or prevent one or more symptoms of deleterious or excessive platelet aggregation relative to an untreated control subject are provided. Thus, LYST inhibitors can be used to treat or prevent diseases or disorders associated with undesirable or deleterious platelet function. Examples of disorders resulting from inappropriate/deleterious activation of platelets include platelet hyper-aggregation or increased mean platelet volume (MPV) and thrombocytosisl arterial thrombosis; venous thrombosis (Budd-Chiari syndrome; Cavernous sinus thrombosis; Cerebral venous sinus thrombosis; deep vein thrombosis; Paget-Schroetter disease; portal vein thrombosis; jugular vein thrombosis; renal vein thrombosis); and microcirculatory thrombosis. Arterial thrombosis can partially or completely obstruct the flow of blood causing downstream ischemia, stroke or myocardial infarction.

Increased or uncontrolled over-expression of platelet derived growth factor (PDGF) and uncontrolled PDGF signaling has been implicated in the growth, angiogenesis and metastasis of cancer cells as well as diseases associated with such as development, survival and metastasis of tumors (reviewed in Andrae, et al., Genes Dev., 22:pp1276-1312 (2008)). Inhibition of PDGF has been shown to reduce glioma cell growth. Several inhibitors of PDGFR-mediated signaling, such as imatinib, have entered clinical trials for treatment of a variety of malignancies. Accordingly, one or more LYST inhibitors can be used to treat or prevent any disease associated with increased or up-regulated expression of PDGF and/or uncontrolled PDGF signaling. Exemplary diseases include cancers such as glioblastomas (e.g., anaplastic oligodendrogliomas) and sarcomas (e.g., dermal sarcoma; esophageal squamous cell sarcomas), as well as retinal vascular disease (e.g., ischemic retinopathies).

B. Methods of Treatment

Inhibition of LYST can be used as a therapeutic mechanism through either local or systemic delivery. In some embodiments, the compositions are administered systemically. Delivery vehicles can be selected and used to target the inhibitors to a particular location or cell type. In other embodiments, the inhibitors are directly administered to the vasculature using a device or graft, such as those discussed above. In further embodiments, the route of administration targets the inhibitors directly to a specific organ or to the local site of injury.

It has been established that the processes of vascular proliferation and inflammation are linked. It is believed that LYST moderates immune processes that contribute to proliferative disorders, such as the restenosis process. Early pharmacological intervention can preclude chronic therapy and any potentially adverse side effects associated with chronic therapy. For example, the compositions disclosed herein can reduce or prevent neointima formation, but allow neo-tissue growth to occur. Methods of treatment and prevention of diseases and disorders using the disclosed LYST inhibitors optionally including a delivery vehicle are discussed in more detail below.

Controls

The effect of a LYST inhibitor can be compared to a control. Suitable controls are known in the art and include, for example, untreated cells or an untreated subject. In some embodiments, the control is untreated tissue from the subject that is treated, or from an untreated subject. Preferably the cells or tissue of the control are derived from the same tissue as the treated cells or tissue. In some embodiments, an untreated control subject suffers from the same disease or condition as the treated subject. For example, in some embodiments, one or more of the pharmacological or physiological markers or pathways affected by anti-LYST treatment is compared to the same pharmacological or physiological marker or pathway in untreated control cells or untreated control subjects. For example, anti-LYST treated subjects can be compared to subjects treated with other inhibitors of neointima formation, such as rapamycin. The subjects treated with other inhibitors of neointima formation can have a greater incidence of in post-operative stenosis, or a reduced formation of neo-vascular tissue than do subjects treated with the LYST inhibitors.

Pharmaceutical compositions including one or more LYST inhibitors can be administered in a variety of manners, depending on whether local or systemic treatment is desired, and depending on the area to be treated. For example, the disclosed compositions can be administered intravenously, intraperitoneally, intramuscularly, subcutaneously, intracavity, or transdermally. The compositions may be administered parenterally (e.g., intravenously), by intramuscular injection, by intraperitoneal injection, transdermally, extracorporeally, topically or the like, including topical intranasal administration or administration by inhalation.

Parenteral administration of the composition, if used, is generally characterized by injection. Injectable formulations can be prepared in conventional forms, either as liquid solutions or suspensions, solid forms suitable for solution of suspension in liquid prior to injection, or as emulsions. Administration involving use of a slow release or sustained release system, such that a constant dosage is maintained, is also discussed.

In certain embodiments, the compositions are administered locally, for example, by injection directly into a site to be treated. Local delivery of drugs can reduce side effects or toxicity associated with systemic delivery and can result in enhanced treatment outcome due to an increased localized dose.

Methods of administering the disclosed inhibitors of LYST locally (i.e., via local drug delivery, LDD) are provided. In certain embodiments, LYST inhibitors can be administered directly to a treated tissue, such as an artery or vein, without engendering adverse systemic effects. In further embodiments, the compositions are injected or otherwise administered directly to one or more surgical sites. Typically, local injection causes an increased localized concentration of the compositions which is greater than that which can be achieved by systemic administration. In preferred embodiments, the compositions are delivered directly to tissue, prostheses, grafts or medical devices by local injection or topical administration. In some embodiments LYST inhibitors delivered locally result in concentrations that are twice, 10 times, 100 times, 500 times, 1000 times or more than 1000 times greater than that achieved by systemic administration of the same compound. In some embodiments the locally administered LYST inhibitors are steadily released at the site of delivery at a constant rate over a period of time. Preferably, the steady release maintains a desired concentration of the LYST inhibitor at the site of delivery.

The disclosed LYST inhibitors can be administered during a period before, during, or after onset of disease symptoms, or any combination of periods before, during or after onset of one or more disease symptoms. For example, the subject can be administered one or more doses of the composition every 1, 2, 3, 4, 5, 6 7, 14, 21, 28, 35, or 48 days prior to onset of disease symptoms. The subject can be administered one or more doses of the composition every 1, 2, 3, 4, 5, 6, 7, 14, 21, 28, 35, or 48 days after the onset of disease symptoms. In some embodiments, the multiple doses of the compositions are administered before an improvement in disease condition is evident. For example, in some embodiments, the subject receives 1, 2, 3, 4, 5, 6 7, 14, 21, 28, 35, or 48, over a period of 1, 2, 3, 4, 5, 6 7, 14, 21, 28, 35, or 48 days or weeks before an improvement in the disease or condition is evident.

Thus, the disclosed compositions including one or more LYST inhibitors can be administered at different times in relation to a diagnosis, prognosis, surgery or injury depending on the disease or disorder to be treated. The timing of commencement of anti-LYST therapy should be determined based upon the needs of the subject, and can vary from at the time of surgery or injury, to one or more days, weeks or months after surgery or injury. Methods for using formulations for delayed release of LYST inhibitors are provided. In some embodiments, therapy using inhibitors of LYST can be discontinued once vascular neo-tissue growth has occurred.

In some embodiments a single dose of one or more inhibitors of LYST is delivered to a subject as one or more bolus doses to raise the blood concentration of the one or more inhibitors to a desired level. The bolus can be given by any means, such as via injection. The placement of the bolus dose can be varied depending upon the desired effect and the target organ or tissue to be treated. In a particular embodiment, a bolus is given prior to the administration of other dosage forms, such as pulsatile release dosages forms. Thus, the desired blood concentration of one or more LYST inhibitors can be maintained for a desired period of time using a combination of formulations for immediate, delayed or pulsatile release.

In the case of adhesions, the deposition of connective tissue between normal anatomical structures is unnecessary and, thus, preventing the development of vascular tissue is not detrimental. Thus, for prevention and reduction of adhesions, LYST inhibitors would typically be applied at the time of surgery or shortly thereafter, for example, within one week. In the case of surgical procedures at risk of neointima formation, for example, stenosis, or restenosis of tissue grafts or stents, one or more LYST inhibitors can be administered at the time of surgery. In the case of injury or surgery involving the skin, the LYST inhibitors can be applied following re-epithelialization of the skin's surface.

Coating onto Grafts and Devices

One or more LYST inhibitors can be delivered locally by incorporating the one or more inhibitors into a medical device such as a stent or other prosthesis, by loading the inhibitor(s) into or onto a structural or sealing material of the device. The rate of release of the inhibitor(s) may be controlled by a number of methods including varying one or more of the ratio of the absorbable material to the agent, the molecular weight of: the absorbable material, the composition of the inhibitor(s), the composition of the absorbable polymer, the coating thickness, the number of coating layers and their relative thicknesses, the inhibitor concentration, and/or physical or chemical binding or linking of the inhibitor(s) to the device or sealing material. Top coats of polymers and other materials, including absorbable polymers may also be applied to control the rate of release of the one or more inhibitors.

In some embodiments, the amount of LYST inhibitor present on a device or graft tissue can be adjusted by changing the delivery vehicle. The penetration of the inhibitor throughout the tissue can also be adjusted. In this way the amount of drug locally released at the site of implantation can be carefully controlled. Typically, the LYST inhibitor or a delivery vehicle carrying the inhibitor is contacted with a device or graft material ex vivo. The contacting can occur in the absence or presence of mild agitation, or other methods known in the art to insure that inhibitor attaches to or penetrates the device or graft tissue. Agitation may be accomplished, for example, by incubation on an orbital shaker, or by vertical rotation, such as by incubation in a vertical carousel of a hybridization oven. The incubation protocol can be varied to affect the positioning of the particles on the device or graft. The amount and localization of attachment of delivery vehicles such as particles to the device or graft can also be varied by varying the type and density of attachment and targeting ligands, such as those described, presented on the vehicle. Exemplary compositions and methods for delivering drugs to vascular grafts ex vivo are discussed in U.S. Published Application Nos. 2006/0002971, 2010/0151436, and U.S. Pat. No. 7,534,448. The compositions and methods can be used to locally deliver LYST inhibitors to grafts with, or without the requirement for further invasive procedures, such as placement of a vascular graft or stent.

C. Dosages and Effective Amounts

In some in vivo approaches, the compositions of LYST inhibitors are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease or disorder, and the treatment being effected.

For all of the disclosed compounds, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally dosage levels of between 0.001 and 100 mg/kg of body weight daily are administered to mammals, most preferably, humans. Generally, for intravenous injection or infusion, dosage may be lower. Preferably, the compositions are formulated to achieve a LYST inhibitor serum level of between about 1 and about 1000 μM at the site where treatment is desired.

In some embodiments, the LYST inhibitors are effective to prevent the normal biological activities of immune cells, such as macrophages, platelets and NK cells. For example, one or more inhibitors can be in an amount effective to reduce or prevent the migratory or chemotactic activity of macrophages.

In one embodiment the one or more LYST inhibitors are in an amount effective to prevent or reduce neointima formation in a subject. In a preferred embodiment the amount of one or more LYST inhibitors does not prevent wound healing or the formation of neotissue in a subject compared to an untreated control. Preferably, the amount of one or more LYST inhibitors is effective to prevent or reduce neointima formation and enhance wound healing in a subject compared to an untreated control.

In another embodiment, the one or more LYST inhibitors are in an amount effective to decrease the amount of blood vessel growth at the site of an injury or surgery, to prevent or decrease the formation of high density cellular and connective tissue that give rise to fibrosis, scarring, keloids or adhesions. Preferably the amount of one or more LYST inhibitors does not prevent wound healing.

In another embodiment, one or more LYST inhibitors are in an amount effective to decrease or inhibit platelet activation. For example, LYST inhibitors can be in an amount effective to prevent or inhibit inappropriate platelet aggregation and secretion of chemokines at the site of an injury or surgery. In a further embodiment, the one or more LYST inhibitors are in an amount effective to decrease the amount of Platelet Derived Growth Factor (PDGF) and/or Transforming Growth Factor Beta (TGFβ) produced or secreted by cells in response to activation by thrombin at a the site of an injury or surgery. One or more LYST inhibitors can be effective to reduce the production or secretion of PDGF-A, PDGF-B, PDGF-C, or PDGF-D. Thus, one or more LYST inhibitors can be effective to reduce the amount of biologically active PDGF in the serum. For example, the amount of PDGF-AA, PDGF-BB, PDGF-CC, PDGF-DD or PGDF-AB can be reduced relative to the amount in an untreated control. Accordingly, one or more LYST inhibitors can be effective to reduce or prevent one or more biological activities that occur as a result of PDGF, or as a result of downstream signaling events controlled by PDGF. For example, one or more LYST inhibitors can be effective to reduce or prevent the activation of one or more tyrosine kinase receptors, such as PDGF Receptor a (PDGFRα); PDGF Receptor (3 (PDGFRβ); and/or PDGF Receptor αβ (PDGFαβ). By reducing or preventing the activity of one or more PDGFs, the LYST inhibitors can reduce or prevent PDGF-mediated induction of several signaling pathways controlling cellular activities including cellular proliferation, chemotaxis and actin reorganization.

In another embodiment, the one or more LYST inhibitors are in an amount effective to prevent or decrease the formation of high density cellular and connective tissue that give rise to fibrosis, scarring, keloids or adhesions. Preferably the amount of one or more LYST inhibitors does not prevent wound healing.

D. Combination Therapies

The disclosed compositions, devices, and grafts including LYST inhibitors can be administered alone, or in combination with one or more additional active agent(s), as part of a therapeutic or prophylactic treatment regime. For example, the composition can be administered on the first, second, third, or fourth day, or combinations thereof. The composition can be administered on the same day, or a different day than the second active agent.

The term “combination” or “combined” is used to refer to either concomitant, simultaneous, or sequential administration of two or more agents. Therefore, the combinations can be administered either concomitantly (e.g., as an admixture), separately but simultaneously (e.g., via separate intravenous lines into the same subject), or sequentially (e.g., one of the compounds or agents is given first followed by the second).

The additional therapeutic agents can be other anti-neointima agents, chemotherapeutic agents, antibodies, antibiotics, antivirals, steroidal and non-steroidal anti-inflammatories, conventional immunotherapeutic agents, immune-suppressants, cytokines, chemokines and/or growth factors, anti-proliferatives or anti-migration agents designed for treating or preventing neointima formation or restenosis, agents which affect migration and extracellular matrix production, agents which affect platelet deposition or formation of thrombus, and agents that promote vascular healing and re-endothelialization, described in Tanguay et al. Current Status of Biodegradable Stents, Cardiology Clinics, 12:699-713 (1994), J. E. Sousa, P. W. Serruys and M. A. Costa, Circulation 107 (2003) 2274 (Part I), 2283 (Part II), K. J. Salu, J. M. Bosmans, H. Bult and C. J. Vrints, Acta Cardiol 59 (2004) 51.

Exemplary anti-thrombin agents include, but are not limited to, Heparin (including low molecular heparin), R-Hirudin, Hirulog, Argatroban, Efegatran, Tick anticoagulant peptide, and Ppack.

Exemplary antiproliferative agents include, but are not limited to, Paclitaxel (Taxol), QP-2 Vincristin, Methotrexat, Angiopeptin, Mitomycin, BCP 678, Antisense c-myc, ABT 578, Actinomycin-D, RestenASE, 1-Chlor-deoxyadenosin, PCNA Ribozym, and Celecoxib.

Exemplary agents modulating cell replication/proliferation include targets of rapamycin (TOR) inhibitors (including sirolimus, everolimus and ABT-578), paclitaxel and antineoplastic agents, including alkylating agents (e.g., cyclophosphamide, mechlorethamine, chlorambucil, melphalan, carmustine, lomustine, ifosfamide, procarbazine, dacarbazine, temozolomide, altretamine, cisplatin, carboplatin and oxaliplatin), antitumor antibiotics (e.g., bleomycin, actinomycin D, mithramycin, mitomycin C, etoposide, teniposide, amsacrine, topotecan, irinotecan, doxorubicin, daunorubicin, idarubicin, epirubicin, mitoxantrone and mitoxantrone), antimetabolites (e.g., deoxycoformycin, 6-mercaptopurine, 6-thioguanine, azathioprine, 2-chlorodeoxyadenosine, hydroxyurea, methotrexate, 5-fluorouracil, capecitabine, cytosine arabinoside, azacytidine, gemcitabine, fludarabine phosphate and aspariginase), antimitotic agents (e.g., vincristine, vinblastine, vinorelbine, docetaxel, estramustine) and molecularly targeted agents (e.g., imatinib, tretinoin, bexarotene, bevacizumab, gemtuzumab ogomicin and denileukin diftitox).

Exemplary anti-restenosis agents include, but are not limited to, immunomodulators such as Sirolimus (Rapamycin), Tacrolimus, Biorest, Mizoribin, Cyclosporin, Interferon .gamma.lb, Leflunomid, Tranilast, Corticosteroide, Mycophenolic acid and Biphosphonate.

Exemplary anti-migratory agents and extracellular matrix modulators include, but are not limited to Halofuginone, Propyl-hydroxylase-Inhibitors, C-Proteinase-Inhibitors, MMP-Inhibitors, Batimastat, Probucol. Examples of antiplatelet agents include, but are not limited to, heparin.

In some embodiments the additional therapeutic agent is N-3,4-trihydroxybenzamide or a pharmaceutically acceptable salt or ester thereof, didox, imidate, or hydroxyurea as described in U.S. Pat. No. 8,029,815.

The additional therapeutic agents can be administered locally or systemically to the subject, or coated or incorporated onto, or into a device or graft. The additional therapeutic reagents can be administered by the same, or by different routes and by different means. For example, one or more LYST inhibitors can be delivered locally combined with one or more of paclitaxel, taxotere and other taxoid compounds, methotrexate, anthracyclines such as doxorubicin, everolimus, serolimus, rapamycin or rapamycin derivatives delivered by different means, such as systemically.

In further embodiments, one or more LYST inhibitors may be used to reduce or prevent the unwanted side-effects associated with the use of one or more additional therapeutic agents. For example, one or more LYST inhibitors can be used with the anti-neoplastic agent bleomycin, to prevent pulmonary fibrosis associated with bleomycin.

The present invention will be further understood by reference to the following non-limiting examples.

### EXAMPLES

### Example 1

### The Beige Mutation Reduces Stenosis in Tissue Grafts

Methods and Materials

Tissue engineered vascular grafts (TEVG) were developed by seeding autologous bone marrow-derived mononuclear cells onto a biodegradable tubular scaffold fabricated from a polyglycolic acid-fiber tube coated with a 50:50 copolymer of poly lactic acid and polycaprolactone.

A murine model for evaluating TEVG function was developed in an effort to elucidate the cellular and molecular mechanisms underlying the formation of TEVG. The inferior vena cava (IVC) interposition graft model is a validated model for investigating the use of vascular grafts in a low pressure, high flow circulatory system similar to the Fontan circulation. This model was used extensively to study neotissue formation in the TEVG.

A biodegradable conduit graft composed of PGA-PCL/LA was implanted into the inferior vena cava of wild-type mice and beige mutant mice, respectively, without cell seeding. Grafts were explanted 2 weeks after surgery, sectioned and measured for the diameter of the vessel and assessed for patency.

Results

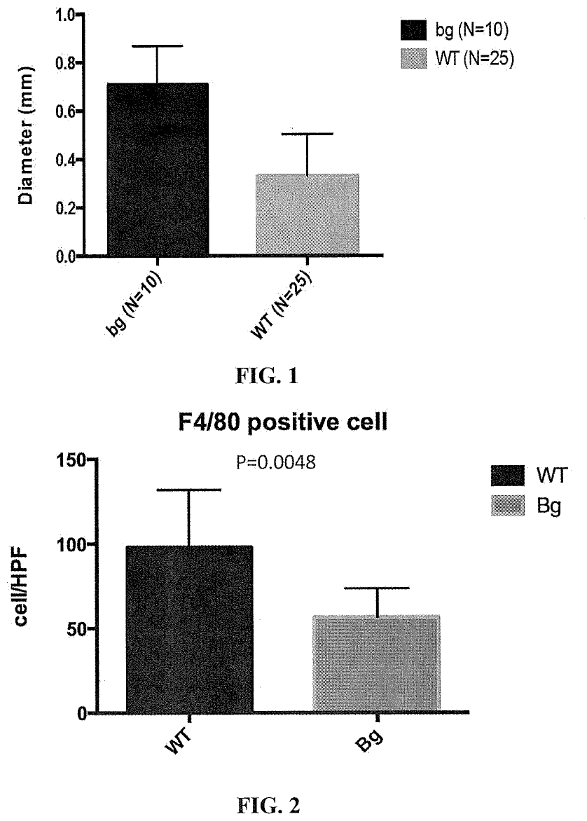

Significant variation in the stenosis rates was observed when TEVG were implanted as IVC interposition grafts, depending on the strain of the mouse. Specifically, it was noted that TEVG implanted in mice with a beige mutation had exceptionally low rates of stenosis. The patency and luminal diameter of TEVG implanted as IVC interposition grafts were compared in both C57B6 (wild-type mice) (N=25) and beige mutants (N=10) (Table 1). The wild-type mice exhibited a 80% stenosis with luminal diameters measuring 0.33 mm whereas the beige mice exhibited a 10% stenosis rate with luminal diameters measuring 0.71 mm (T-test<0.001 and Chi-square 0.0002) (FIG. 1).

TABLE 1
Differences in the patency and luminal diameters of TEVG
explanted from Beige (Bg) and wild-type (Wt) mice.

| | | | | Patent | Patency |
|---|---|---|---|---|---|
| Gunze 21 G | n | Diameter | SD | (>0.45 mm) | (%) |
| Beige | 10 | 0.71 | 0.16 | 9 | 90 |
| Lyst-tm1b | 5 | 0.73 | 0.17 | 5 | 100 |
| WT | 25 | 0.33 | 0.17 | 5 | 20 |

### Example 2

### The beige Mutation Reduces Macrophage Infiltration Into Tissue Grafts

Methods and Materials

It has previously been demonstrated using the murine model that vascular neotissue formation arises from cells derived from the host and that the process of vascular neotissue formation is orchestrated by the immune system, specifically host-derived macrophages. Excessive macrophage infiltration leads to stenosis, while inhibition of macrophage infiltration prevents neotissue formation.

The number of macrophages in the explanted grafts described above in Example 1 was measured for both beige and wild type mice. Immunohistochemistry was carried out on tissue sections of each graft to characterize the number and morphology of endothelial cells in each group, using an antibody specific for the CD31 cell marker.

Results

The TEVG implanted in the beige mice exhibited significantly less macrophage infiltration than TEVG implanted in the wild type mice at 2 weeks after surgery. See FIG. 2. Notably, immunohistochemistry for endothelial cells (using an antibody specific for CD 31) did not show any difference between graft sections from wild-type and beige mice, indicating that neotissue formation was similar in both groups.

### Example 3

### The Beige Phenotype Arises From a Mutation in the LYST Gene

Methods and Materials

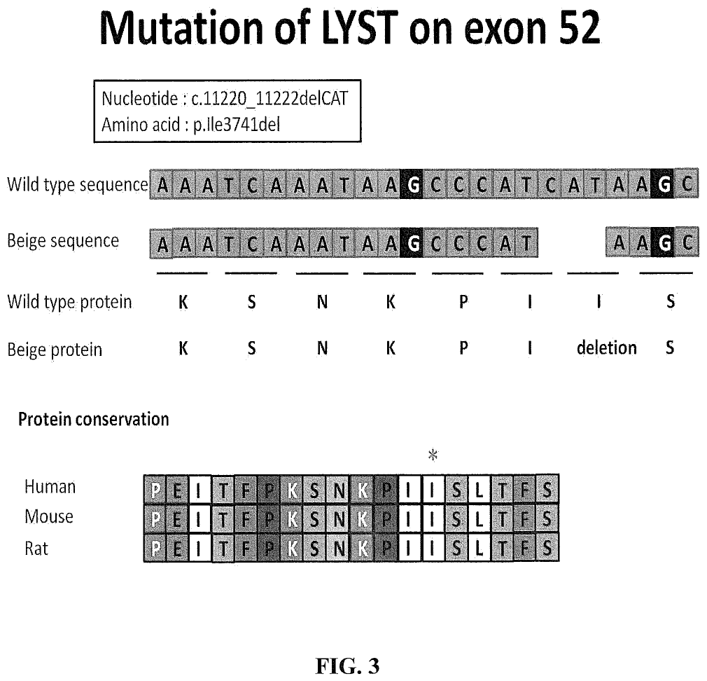

The beige mutation arises from a spontaneous mutation of the LYST gene (FIG. 3). For analysis of protein expression, 6 different primers, designated LYST1, LYST3, LYST4, LYSTS and LYST6, respectively, were designed to cover the complete LYST gene region. The LYST-5 primer covered the area of exon 52 that included the amino acid mutation identified in the beige mouse genotype. Raw cells were used as a standard to calculate relative mRNA levels.

Results

The specific mutation responsible for the beige phenotype in Beige mice was identified. The full length LYST gene encodes a 3801 amino acid polypeptide. The mutation of LYST occurred on exon 52 resulting in deletion of the Amino acid isoleucine (i1e3741de1) (FIG. 3).

Molecular analyses demonstrated that there was no significant difference in expression of mRNA between both groups. Despite equivalent levels of transcription of the wild-type and mutant proteins, there was no translation of the mutant protein.

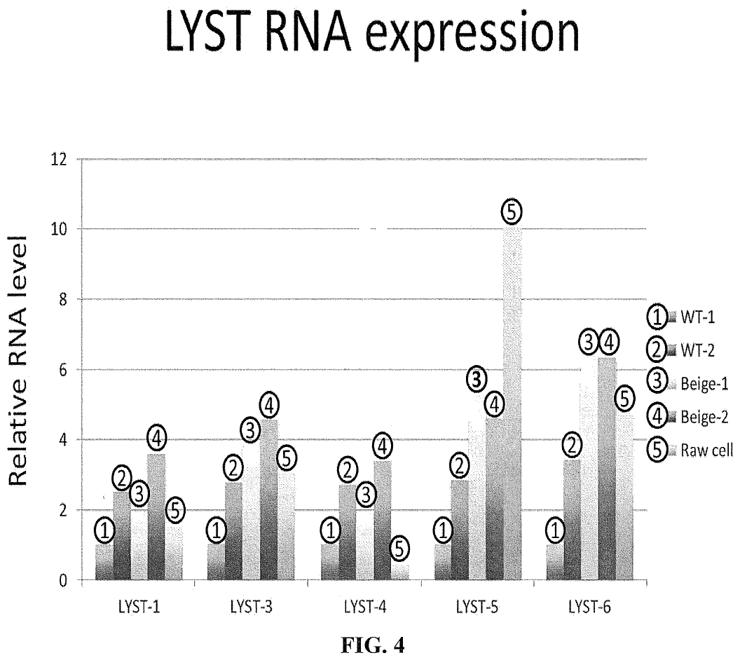

Further, LYST protein expression was depleted in beige mutant mouse by immunohistochemistry and western blotting (FIG. 4). These results indicated that LYST gene mutation in beige mouse affect protein modification level after translation from gene.

### Example 4

### The beige Mutation in the LYST Gene Alters Immune Processes

Methods and Materials

The hypothesis that the beige mutation inhibits the formation of TEVG stenosis by modulating the immune system was tested. Bone marrow was transplanted from C57B6 (wild-type) mice into beige mutant mice, and vice versa. FACS analyses were carried out to determine the percentage of LYST monocytes in Beige and wild-type mice. In addition, the ability of platelet activation upon stimulation with fibrin was reduced in beige mutant mice compared with wild-type.

Results

Significant inhibition of TEVG stenosis was demonstrated in wild-type mice transplanted with bone marrow from the beige mice. Similarly, when TEVG were implanted in beige mice transplanted with the bone marrow from wild-type mice they exhibited high rates of TEVG stenosis. These findings indicated the beige mutation inhibits TEVG stenosis via its effect on the immune system. FACS analysis of monocytes and macrophages from Wt and Beige mice, respectively, demonstrated difference in the phenotype population. A series of scatter dot plots showing representative results of analytical flow cytometry show the proportion of monocyte cells stained for CD115 (Y-axis) and CD1lb (X-axis) from WT3, WT4, bg3 and bg4 mice, respectively. The distribution of cells that are stained for LYC6 and F4/80 from WT3, WT4, bg3 and bg4 mice, respectively, were also determined. The percentage of cells designated as LYC6 “Hi” and LYC6 “lo” was calculated. Results are representative of peritoneal macrophages 3 days after stimulation with thioglycolate, gated to exclude dead cells and debris.

Scatter dot plots showed representative results of analytical flow cytometry, plotting cells stained for PE-A (Y-axis) against FITC-A fluorescence (X-axis) from WT mice and Beige mice, respectively. Cells were gated to exclude dead cells and debris following platelet activation following stimulation with fibrin. The number of LY6C “hi” monocytes was higher in wild type group (WT3, WT4), whereas the number of Ly6C “low” monocytes was higher in beige group (bg 3, bg) in peritoneal macrophage at 3 days after stimulation with thioglycolate.

FACS analysis also demonstrated that the ability of platelet activation upon stimulation of cells with fibrin was reduced in beige mutant mice, as compared with wild-type mice.

### Example 5

### LYST-Mediated Immunomodulation Can be Achieved Using an Antibody to the LYST Protein

Methods and Materials

The effects of blocking the LYST protein were investigated using an anti-LYST antibody. C57B6 mice were treated with the anti-LYST antibody and then implanted with TEVG. Anti-LYST antibody was injected into peritoneum of wild type mouse in each of three different treatment groups (0 mg/kg (control), 10 mg/kg, or 50 mg/kg) at 1 day before surgery, as well as 1 week after surgery. Mice were sacrificed and graft and spleen was explanted at 2 weeks after surgery. Histology was carried out using Hematoxylin and eosin staining (HE stain) of the graft. Spleen tissue was analyzed by Western blotting using an antibody specific for the LYST protein to determine the relative effective amount of LYST protein in each treatment group.

Results

The systemic injection of anti-LYST antibody showed improvement of graft patency in dose dependent fashion. A noted inhibition of macrophage infiltration and decreased TEVG stenosis was observed. This dose-dependent increase in patency was accompanied by a dose-dependent reduction in the presence of the LYST protein within spleen tissue, as determined by immunohistochemistry. Thus, a method of immunomodulation that results in improved neotissue formation and inhibition of the formation of TEVG stenosis has been established.

### Example 6

### Transgenic Mice Lacking a Functional Lyst Gene do Not Exhibit Stenosis of TEVG

Methods and Materials

C57BL/6 transgenic mice lacking a functional LYST protein were generated. These mice were designated C57BL/6 Lyst^{tm1b}. The technology used to generate this knockout strain uses a LacZ reporter. PCR for LacZ confirmed the knockout model.

To determine whether C57BL/6Lysttmlb knockout mice would develop stenosis following TEVG implantation, 5 C57BL/6 Lysttmlb mice and 5 C57BL/6 wild type mice were implanted with unseeded TEVG. The TEVG were explanted and examined 2 weeks following implantation.

Results

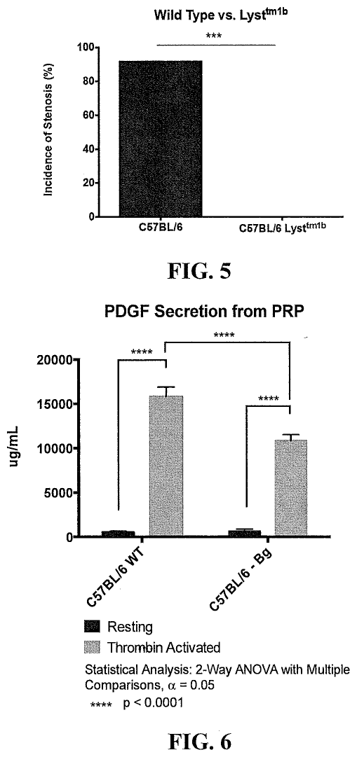

100% of unseeded tissue engineered vascular grafts (TEVG) implanted in Lyst^{tm1b} knockout mice (n=5) were patent after 2-week implantation (see FIG. 5; Table 1). In contrast, C57BL/6 wild type mice exhibited approximately 90% incidence of stenosis.

### Example 7

### Lyst Modulates Platelet Function in Response to Activation

Methods and Materials

The Lyst protein is understood to be involved in platelet function, and Chediak Higashi Syndrome, the human correlate, is characterized as a “platelet storage pool deficiency.” Platelet rich plasma (PRP) was obtained from Beige (Bg) and Wild Type (WT) mice. Samples were activated by thrombin and total concentrations of secreted PDGF (platelet derived growth factor) from each group (n=7) were compared to resting, non-activated controls (n=5).

Results

In both the activated and resting groups, thrombin activation resulted in significantly greater concentrations of PDGF. However, the PDGF secretion from activated platelets was significantly decreased in the Beige group when compared to Wild Type (see FIG. 6). Thus, reduction of Lyst activity also reduced PDGF, indicating that agents which reduce Lyst activity can also function as anti-platelet agents.

1-24. (canceled)

25. A method of reducing or preventing macrophage infiltration, scar formation, or stenosis in a subject, comprising
administering to a subject up to three days prior to surgery, at the time of surgery, or up to 14 days after surgery an effective amount of a composition comprising:
a) an inhibitor of lysosomal trafficking regulator (LYST) inhibiting LYST transcript or LYST protein, in an amount effective to reduce or prevent macrophage infiltration, natural killer cell activation, and to reduce or prevent platelet activation in the subject; and
b) a physiologically acceptable carrier,
wherein the amount of the LYST inhibitor does not prevent vascular neotissue formation in the subject,
optionally in combination with one or more additional therapeutic agents,
wherein the LYST inhibitor is administered in an amount and at a time effective to decrease scar formation, myocardial infarction, scarring adhesions, and liver fibrosis or to reduce or prevent platelet activation that could lead to arterial or venous thrombosis in a subject.

26. The method of claim 25, wherein the composition is in a dosage effective to reduce or prevent macrophage infiltration.

27. The method of claim 25, wherein the composition is in a dosage effective to reduce or prevent platelet activation.

28. The method of claim 25, wherein the composition further comprises a delivery vehicle selected from the group consisting of nanoparticles, microparticles, micelles, emulsions, synthetic lipoprotein particles, liposomes, carbon nanotubes, gels, or coatings.

29. The method of claim 25, wherein the one or more additional therapeutic agents are selected from the group consisting of anti-neointima agents, chemotherapeutic agents, steroidal and non-steroidal anti-inflammatories, conventional immunotherapeutic agents, immune-suppressants, cytokines, chemokines, and growth factors.

30. The method of claim 25, wherein the composition is administered with a vascular graft or medical device.

31. The method of claim 30 wherein the composition is coated onto or incorporated into the graft or device.

32. The method of claim 30, wherein the medical device is selected from the group consisting of stents, implants, needles, cannulas, catheters, shunts, balloons, and valves.

33. The method of claim 30, wherein the medical device is a stent.

34. The method of claim 33, wherein the medical device is a drug eluting stent that elutes the composition.

35. The method of claim 30, wherein the vascular graft is an autologous, preserved autologous, allogeneic, xenogenic or synthetic graft.

36. The method of claim 25, comprising administering to the subject in need thereof one or more additional therapeutic agents.

37. The method of claim 25 wherein the subject is at risk of or has restenosis or other vascular proliferation disorder.

38. The method of claim 25 wherein the subject has undergone or is undergoing vascular trauma, angioplasty, vascular surgery, or transplantation arteriopathy.

39. The method of claim 25, wherein the composition is used to reduce or prevent the formation of scar tissue, promote healing, reduce or prevent the development of hypertrophic scarring, keloids, or adhesions, reduce or prevent fibrosis of the liver, fibrosis of the lungs, fibrosis of the heart or fibrosis of the kidneys, reduce or prevent neointima formation, stenosis or restenosis, reduce or prevent thrombosis, or any combination thereof in a subject relative to an untreated control subject.

40. The method of claim 25, wherein reducing or preventing the formation of scar tissue promotes integration but blocks encapsulation of one or more bio-prosthesis devices selected from the group consisting of pacemakers, nerve stimulators, replacement heart valves and artificial joints.

41. The method of claim 25, wherein reducing or preventing the formation of scar tissue is effective to treat or prevent neointima formation at a site of implantation of a vascular implant, a site of vascular injury, or a site of surgery in a subject, relative to an untreated control subject.

42. The method of claim 37, wherein the composition is used to reduce the expression of platelet derived growth factor, transforming growth factor beta, or combination thereof in a subject relative to an untreated control subject.

43. A method of reducing stenosis or restenosis of a vascular graft comprising treating the graft ex vivo with a composition comprising:
a) an inhibitor of lysosomal trafficking regulator (LYST) inhibiting LYST transcript or LYST protein, in an amount effective to reduce or prevent macrophage infiltration, natural killer cell activation, and to reduce or prevent platelet activation in the subject; and
b) a physiologically acceptable carrier,
wherein the amount of one or more inhibitors of LYST does not prevent vascular neotissue formation in the subject following graft implantation,
prior to implantation of the graft into a subject.
